(19) United States
(12) Patent Application Publication
(10) Pub. No.: US 2026/0355228 A1
(43) Pub. Date: Nov. 12, 2026

(54) TREATMENT OF FAMILIAL ADENOMATOUS POLYOPSIS USING A 13-MEMBERED MACROLIDE

(71) Applicant: ZIKANI THERAPEUTICS, INC., Watertown, MA (US)

(72) Inventors: Vasudeo BADARINARAYANA, Acton, MA (US); Yoshitaka ICHIKAWA, Laguna Niguel, CA (US); Roger B. CLARK, Mesa, AZ (US); Shuhao SHI, Waltham, MA (US); Vijay MODUR, Acton, MA (US)

(21) Appl. No.: 19/107,101

(22) PCT Filed: Aug. 31, 2023
(86) PCT No.: PCT/US2023/031687

§ 371 Date: Feb. 27, 2025

Related U.S. Application Data
(60) Provisional application No. 63/403,130, filed on Sep. 1, 2022.

(51) Int. Cl. A61K 31/7072; A61K 31/7056; A61P 1/00; A61P 35/00

(52) U.S. Cl. CPC A61K 31/7072; A61K 31/7056; A61P 1/00; A61P 35/00

(57) ABSTRACT

Disclosed herein is a method of treating FAP to a patient in need of such treatment, comprising administering to the patient a compound of formula I: or a pharmaceutically acceptable salt thereof, wherein R₉ₐ is selected from the group consisting of H, C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, C(═O)cycloalkyl, and C(═O)NH-aryl; and R₁₀ₐ is a heteroaryl.

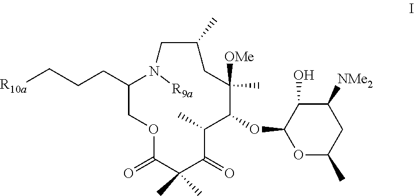

### FIELD OF THE INVENTION

The invention relates to a method of treating familial adenomatous polyposis using macrolide compounds.

### BACKGROUND OF THE INVENTION

Familial adenomatous polyposis (FAP) is characterized by the development of numerous (100-1,000) non-malignant adenomas in the colon and rectum starting in adolescence that eventually progress to colorectal cancer in mid-life if left untreated. This is commonly associated with clinical symptoms such as rectal bleeding and resultant anemia in ~40% of patients [1]. The primary driver of the disease is deficient adenomatous polyposis coli (APC) protein activity resulting from germline mutations in the gene. The mutations are present in 80% of FAP patients and inherited in an autosomal dominant pattern [2-4]. Mutations can be insertions, deletions or nonsense which result in loss of function of the APC protein and are primarily located in the 5′ region of the 8.5-kb coding region [5, 6]. Premature stop codons in the APC gene are generally caused by nonsense (30%) or frameshift (68%) mutations resulting in the synthesis of a truncated, dysfunctional protein and is causative in the majority of FAP patients [2-5]. In addition to colorectal carcinoma, the absence of a functional APC protein is linked to a predisposition for other diseases such as desmoid tumors and Turcot syndrome, which is associated with brain tumors [7].

The APC gene encodes for a large 312 kDa protein composed of multiple functional domains that are involved with cell division, adhesion and cellular polarization during embryonic development [8, 9]. The general structure of the protein consists of 4 domains which permit the protein to interact and form active complexes with other proteins such as b-catenin, axis inhibition protein (AXIN) and microtubule plus-end binding protein (EB1) [8-10]. APC acts as a tumor suppressor gene and the protein negatively regulates the b-catenin/wnt-signaling pathway by mediating the degradation of b-catenin in the cytoplasm [11]. The central region of APC between codon 1284 and codon 1580 is referred to as the mutation cluster region (MCR) and approximately 60% of all APC mutations occur in this area, with a majority being nonsense or frameshift mutations generating a truncated protein [2, 12, 13]. The MCR region is involved with b-catenin ubiquitination and in the absence of this function, cytoplasmic levels of b-catenin increase [8, 9]. Subsequently, b-catenin is translocated to the nucleus resulting in elevated nuclear levels and increased activation of b-catenin/wnt-pathway genes such as c-myc and other proto-oncogenes [14-16]. Somatic mutations in APC gene occur in 80% of colon cancers of which, 30% are nonsense [3, 12]. These somatic mutations are a known to play a critical role in cancer initiation [12, 17].

The APC^{min} (multiple intestinal neoplasia) mouse model of FAP was developed in C57BL/6 mice by exposure to the mutagen N-ethyl-N-nitrosourea and is a widely used model of the disease [18, 19]. Pathology is due to a nonsense mutation at codon 850 of the murine homolog of the APC gene resulting in a nonfunctional, truncated protein [18, 20]. This mutation is similar to the mutations in the APC gene carried in human FAP patients [21]. The APC^{min} model utilizes mice that are heterozygous for the mutation (homozygous APC^{min} mice are nonviable) having an average lifespan of 119 days with chronic anemia caused by intestinal polyps as the major causative factor of lethality [18]. Moribund APC^{min} mice present with numerous (~30/GI tract) adenomas, mainly located in the small intestines [18-20]. Adenomas are detectable in APC^{min} mice at 5 weeks of age and these early lesions appear as crypts crowded with cells of similar morphology observed in low grade colonic lesions found in FAP patients [19, 21]. Accumulation of nuclear b-catenin can be observed in adenomas in APC^{min} mice by immuno-histochemistry [19]. Additionally, disruption of nuclear translocation of b-catenin leads to reduced expression of genes that are transcriptionally co-regulated by b-catenin such as c-myc resulting in decreased polyp formation in APC^{min} [22].

Macrolides and other ribosome targeting antibiotics have been shown to induce read-through of premature stop codons in eukaryotic cells leading to translation of the full-length mRNA. Investigations by Zilberg et al. [23] demonstrated that tylosin can suppress translation termination generated by nonsense mutations in the APC gene using colorectal cancer cell lines and in vivo colon carcinoma models. Other studies have demonstrated that gentamycin and erythromycin treatment also suppressed nonsense mutations in the APC gene in human colon carcinoma cells, inhibited the in vivo growth of human colorectal xenografts possessing APC nonsense mutations; and reduced the number of intestinal polyps in a mouse model of FAP [23, 24]. Subsequently, in a clinical study of FAP patients harboring APC nonsense mutations, Kariv et al. [25] showed that erythromycin resulted in a decrease in adenoma burden. Recent investigations utilizing aminoglycosides, demonstrated that these antibiotics can promote read-through of premature stop codons in the APC and cystic fibrosis transmembrane conductance regulator genes resulting in partial alleviation of the disease state [23]. In general, the degree of full-length, functional protein induced by macrolide and aminoglycoside mediated read-through is in the range of 2-10% of normal levels which has been shown to be sufficient to restore normal cell function [26, 27]. However, the aminoglycoside and macrolide antibiotics that have demonstrated read-through have weak potency, inadequate target tissue exposure and adverse effects that limit chronic administration, thereby precluding their clinical use in FAP therapy [28-30].

As a result, a need remains for novel macrolide compounds to treat FAP.

### SUMMARY OF THE INVENTION

This and other needs are met by the present invention which is directed to a method of treating FAP in a patient in need of such treatment, comprising administering to the patient a compound of formula I:

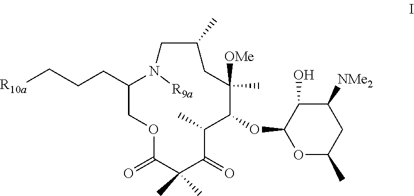

or a pharmaceutically acceptable salt thereof, wherein
R₉ₐ is selected from the group consisting of H or optionally substituted C₁₋₆ alkyl; and
R₁₀ₐ is an optionally substituted heteroaryl.

### BRIEF DESCRIPTION OF THE FIGURES

FIG. 1 summarizes the antiproliferative activity of a compound of the invention in human colon carcinoma cells.

FIG. 2 summarizes the pharmacokinetic parameters of a compound of the invention after oral administration at 100 mg/kg bw in CD1 mice.

FIG. 3 shows that treatment with a compound of the invention reduces nuclear translocation of beta-catenin colon carcinoma cells harboring a nonsense mutation of the APC.

FIG. 4 shows decreased protein levels of c-myc in colon carcinoma cells treated with a compound of the invention.

FIG. 4 shows that long-term survival and decreased anemic phenotype in APC^{min} mice treated with a compound of the invention.

FIG. 5 shows the reduced number of intestinal polyps and dyplasic lesions observed in APC^{min} mice treated with a compound of the invention.

FIG. 7 shows that epithelial cells in the small intestines from a compound of the invention have reduced nuclear levels off beta-catenin.

### DETAILED DESCRIPTION OF THE INVENTION

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety, including U.S. Pat. Publ. No. 2013/0090326. In case of conflict, the present specification, including these definitions, will control.

The terms “a,” “an,” and “the” as used herein not only include aspects with one member, but also include aspects with more than one member. Thus, “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

The term “about” as used herein means “approximately” and is used to modify a numerical value indicates a defined range around that value. If “X” were the value, “about X” would generally indicate a value from 0.95× to 1.05×. Any reference to “about X” specifically indicates at least the values X, 0.95×, 0.96×, 0.97×, 0.98×, 0.99×, 1.0 IX, 1.02×, 1.03×, 1.04×, and 1.05×. Thus, “about X” is intended to teach and provide written description support for a claim limitation of, e.g., “0.98×.” When the quantity “X” only includes whole-integer values (e.g., “X carbons”), “about X” indicates from (X−1) to (X+1). In this case, “about X” as used herein specifically indicates at least the values X, X−1, and X+1.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

The following abbreviations and terms have the indicated meanings throughout:

| Abbreviation | Meaning |
|---|---|
| AcOH | acetic acid |
| APC | Adenomatous polyposis coli |
| br | broad |
| °C | degrees Celsius |
| conc | concentrated |
| CF | Cystic fibrosis |
| d | doublet |
| dd | doublet of doublet |
| dt | doublet of triplet |
| DCM | dichloromethane |
| DIEA or | N,N-di-isopropyl-N-ethylamine |
| DIPEA | |
| DMA | N,N-dimethylacetamide |
| DME | 1,2-dimethoxyethane |
| DMF | N,N-dimethylformamide |
| DMSO | dimethyl sulfoxide |
| dppf | 1,1′-bis(diphenylphosphano)ferrocene |
| EI | Electron Impact ionization |
| equiv | equivalents |
| FAP | Familial adenomatous polyposis |
| FFPE | Formalin fixed, paraffin embedded |
| g | gram(s) |
| GC/MS | gas chromatography/mass spectrometry |
| h or hr | hour(s) |
| HATU | 2-(1H-7-azabenzotriazol-1-yl)-1,1,3,3-tetramethyl |
| | uronium hexafluorophosphate |
| HPLC | high pressure liquid chromatography |
| JEB | Junctional epidermolysis bullosa |
| L | liter(s) |
| LC/MS | liquid chromatography/mass spectrometry |
| M | molar or molarity |
| m | Multiplet |
| MeOH | methanol |
| mg | milligram(s) |
| MHz | megahertz (frequency) |
| min | minute(s) |
| mL | milliliter(s) |
| μL | microliter(s) |
| μM | micromolar |
| μmol | micromole(s) |
| mM | Millimolar |
| mmol | millimole(s) |
| mol | mole(s) |
| MS | mass spectral analysis |
| Ms | mesyl |
| N | normal or normality |
| NBF | Neutral buffered formalin |
| nM | Nanomolar |
| NMR | nuclear magnetic resonance spectroscopy |
| q | Quartet |
| quant | quantitative |
| RDEB | Recessive dystrophic epidermolysis bullosa |
| rt | Room temperature |
| s | Singlet |
| t or tr | Triplet |
| THF | tetrahydrofuran |
| Ts | tosyl |

The symbol “-” means a single bond, “=” means a double bond, “≡” means a triple bond, [symbol] means a single or double bond. The symbol [symbol] refers to a group on a double-bond as occupying either position on the terminus of a double bond to which the symbol is attached; that is, the geometry, E- or Z-, of the double bond is ambiguous. When a group is depicted removed from its parent Formula, the “~” symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied.

The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

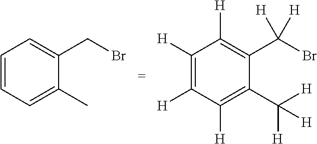

If a group “R” is depicted as “floating” on a ring system, as for example in the following Formula.

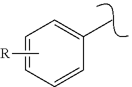

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused or bridged ring system, as for example in the following Formulas.

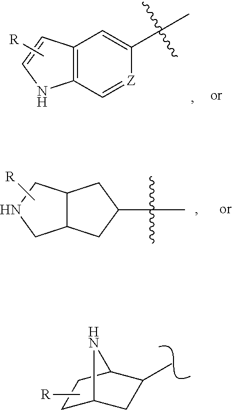

then, unless otherwise defined, a substituent “R” may reside on any atom of the fused or bridged ring system, assuming replacement of a depicted hydrogen (for example the —NH— in the Formula above), implied hydrogen (for example as in the Formula above, where the hydrogens are not shown but understood to be present), or expressly defined hydrogen (for example where in the Formula above, “Z” equals ═CH—) from one of the ring atoms, so long as a stable structure is formed. In the example depicted, the “R” group may reside on either the 5-membered or the 6-membered ring of the fused or bridged ring system.

When a group “R” is depicted as existing on a ring system containing saturated carbons, as for example in the following Formula

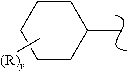

where, in this example, “y” can be more than one, assuming each replaces a currently depicted, implied, or expressly defined hydrogen on the ring; then, unless otherwise defined, where the resulting structure is stable, two “R's” may reside on the same carbon. In another example, two R's on the same carbon, including that carbon, may form a ring, thus creating a spirocyclic ring structure with the depicted ring as for example in the following Formula

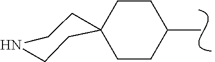

The term “alkyl” as used herein includes an aliphatic hydrocarbon chain that may be straight chain or branched. The chain may contain an indicated number of carbon atoms: For example, C₁-C₁₀ indicates that the group may have from 1 to 10 (inclusive) carbon atoms in it. If not otherwise indicated, an alkyl group contains from 1 to about 20 carbon atoms. In some aspects, alkyl groups have 1 to about 10 carbon atoms. In some aspects, alkyl groups (“lower alkyl”) have 1 to 8, 1 to 6, or 1 to 3 carbon atoms in the chain. Examples may include, but are not limited to, methyl, ethyl, propyl, isopropyl (iPr), 1-butyl, 2-butyl, isobutyl (iBu), tert-butyl, pentyl, 2-methylbutyl, 1,1-dimethylpropyl, hexyl, heptyl, octyl, nonyl, decyl, docecyl, cyclopentyl, or cyclohexyl.

An alkyl group can be unsubstituted or optionally substituted. When optionally substituted, one or more hydrogen atoms of the alkyl group (e.g., from 1 to 4, from 1 to 2, or 1) may be replaced with a moiety independently selected from the group consisting of chloro, fluoro, hydroxy, alkoxy, amino, alkylamino, acylamino, thio, and alkylthio. In some aspects, the alkyl group is unsubstituted or not optionally substituted.

The term “aryl” as used herein includes cyclic aromatic carbon ring systems containing from 6 to 18 carbons. Examples of an aryl group include, but are not limited to, phenyl, naphthyl, anthracenyl, tetracenyl, biphenyl and phenanthrenyl.

An aryl group can be unsubstituted or optionally substituted. When optionally substituted, one or more hydrogen atoms of the aryl group (e.g., from 1 to 5, from 1 to 2, or 1) may be replaced with a moiety independently selected from the group consisting of alkyl, cyano, acyl, halo, haloalkyl, hydroxy, alkoxy, amino, alkylamino, acylamino, thio, and alkylthio. In some aspects, the alkoxy group is unsubstituted or not optionally substituted.

The term “arylalkyl” or “aralkyl” as used herein includes an alkyl group as defined herein where at least one hydrogen substituent has been replaced with an aryl group as defined herein. Examples include, but are not limited to, benzyl, 1-phenylethyl, 4-methylbenzyl, and 1,1-dimethyl-1-phenylmethyl.

An arylalkyl or aralkyl group can be unsubstituted or optionally substituted as per its component groups. For example, but without limitation, the aryl group of an arylalkyl group can be substituted, such as in 4-methylbenzyl. In some aspects, the group is unsubstituted or not optionally substituted, especially if including a defined substituent, such as a hydroxyalkyl or alkylaminoalkoxy group.

As used herein, “fluoroalkyl” includes an alkyl group wherein the alkyl group includes one or more fluoro-substituents. Examples include, but are not limited to, trifluoromethyl.

As used herein, “geminal” substitution includes two or more substituents that are directly attached to the same atom. An example is 3,3-dimethyl substitution on a cyclohexyl or spirocyclohexyl ring.

As used herein, “halo” or “halogen” includes fluoro, chloro, bromo, and iodo.

The term “heteroaryl” or “heterocycloaryl” includes mono and bicyclic groups that are completely unsaturated or partially unsaturated of about 4 to about 14 ring atoms (e.g., 4 to 10 or 5 to 10 atoms) containing at least one heteroatom. Heteroatom as used in the term heteroaryl refers to oxygen, sulfur and nitrogen. A nitrogen atom of a heteroaryl is optionally oxidized to the corresponding N-oxide. Examples include, but are not limited to, pyrazinyl, furanyl, thienyl, pyridyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridine, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, and benzothiazolyl. Other examples include fused heteroaryls such as

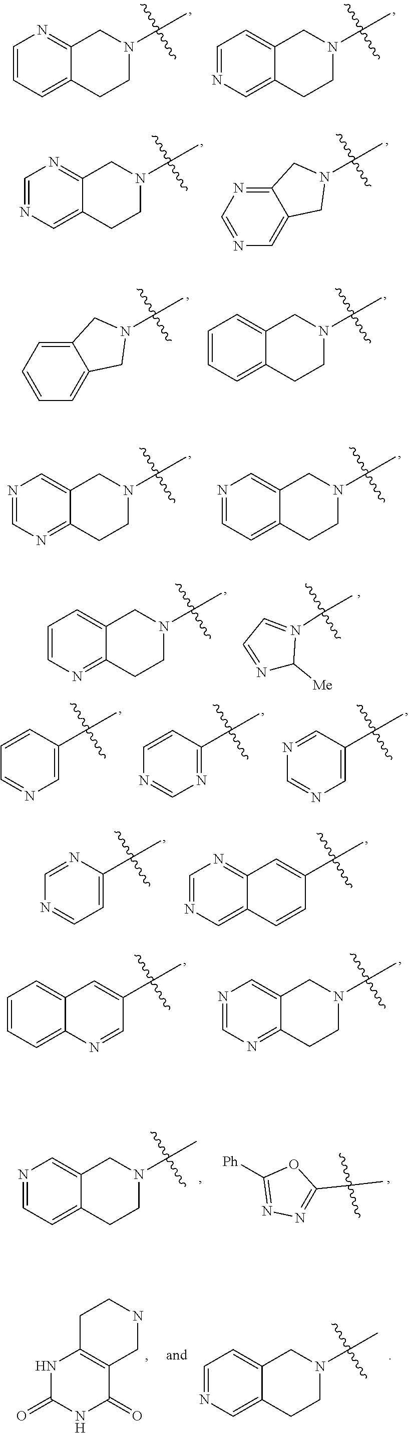

The term “heteroarylene” or “heterocycloarylene” as used herein includes a heteroaryl group that is substituted at two points.

An heteroaryl group can be unsubstituted or optionally substituted. When optionally substituted, one or more hydrogen atoms of the heteroaryl group (e.g., from 1 to 5, from 1 to 2, or 1) may be replaced with a moiety independently selected from the group consisting of alkyl, cyano, acyl, halo, haloalkyl, hydroxy, oxo, alkoxy, amino, alkylamino, acylamino, thio, and alkylthio. In some aspects, the heteroaryl group is unsubstituted or not optionally substituted.

The term “heteroaroyl” as used herein includes a heteroaryl-C(O)— group wherein heteroaryl is as defined herein. Heteroaroyl groups include, but are not limited to, thiophenoyl, nicotinoyl, pyrrol-2-ylcarbonyl, and pyridinoyl.

The term “heterocycloalkyl” may be used interchangeably herein, and as used herein includes a heterocyclyl-C(O)— group wherein heterocyclyl is as defined herein. Examples include, but are not limited to, N-methyl prolinoyl and tetrahydrofuranoyl.

As used herein, “heterocyclyl” (heterocyclo; heterocyclic; heterocycloalkyl) includes a non-aromatic saturated ring of about 3 to about 8 ring atoms (e.g., 5 to about 10 ring atoms, or 3 to about 6 ring atoms), in which one or more of the atoms in the ring system is an element or elements other than carbon, e.g., nitrogen, oxygen or sulfur. A heterocyclyl group optionally comprises at least one sp²-hybridized atom (e.g., a ring incorporating an carbonyl, endocyclic olefin, or exocyclic olefin). In some embodiments, a nitrogen or sulfur atom of the heterocyclyl is optionally oxidized to the corresponding N-oxide, S-oxide or S,S-dioxide. The monocyclic heterocycle means a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyridazin-3(2H)-onyl, pyridin-2(1H)-onyl, pyrrolinyl, pyrrolidinyl, tetrahydrofuranyl, tetrahydropyranyl, tetrahydropyridinyl, tetrahydropyrimidinyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The term “heterocycloalkylene” as used herein includes a heterocyclyl (heterocyclo; heterocyclic) group that is substituted at two points.

The term “heterocyclyl” also includes multicyclic rings such as a bicyclic heterocycle, or a tricyclic heterocycle which may be in a fused, bridged, or spiro orientation. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, 3-azabicyclo[3.1.0]hexane, 3-azabicyclo[4.1.0]heptane, 3-azabicyclo[3.2.0]heptane, (3aR,6aS)-hexahydro-1H-2λ₂-cyclopenta[c]pyrrole, (3aR,7aS)-octahydro-2λ₂-isoindole.

Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms.

A heterocycyl group can be unsubstituted or optionally substituted. When optionally substituted, one or more hydrogen atoms of the group (e.g., from 1 to 4, from 1 to 2, or 1) may be replaced with a moiety independently selected from the group consisting of alkyl, halo, haloalkyl, oxo, acetyl, hydroxy, alkoxy, amino, alkylamino, acylamino, thio, and alkylthio. In some aspects, a substituted heterocycyl group can incorporate an exo- or endocyclic alkene (e.g., cyclohex-2-en-1-yl). In some aspects, the heterocycyl group is unsubstituted or not optionally substituted.

The monocyclic, bicyclic, and tricyclic heterocycles are connected to the parent molecular moiety through any carbon atom or any nitrogen atom contained within the rings, and can be unsubstituted or substituted.

As used herein, the term “hydrophilic moiety” or “hydrophilic group” includes a moiety or a functional group that has a strong affinity to water. Examples may include, but are not limited to, a charged moiety, such as a cationic moiety or an anionic moiety, or a polar uncharged moiety, such as an alkoxy group or an amine group.

As used herein, the term “hydroxyalkyl” includes an alkyl group where at least one hydrogen substituent has been replaced with an alcohol (—OH) group. In certain aspects, the hydroxyalkyl group has one alcohol group. In certain aspects, the hydroxyalkyl group has one or two alcohol groups, each on a different carbon atom. In certain aspects, the hydroxyalkyl group has 1, 2, 3, 4, 5, or 6 alcohol groups. Examples may include, but are not limited to, hydroxymethyl, 2-hydroxyethyl, and 1-hydroxyethyl.

When any two substituent groups or any two instances of the same substituent group are “independently selected” from a list of alternatives, the groups may be the same or different. For example, if R^{a} and R^{b} are independently selected from the group consisting of alkyl, fluoro, amino, and hydroxyalkyl, then a molecule with two R^{a} groups and two R^{b} groups could have all groups be an alkyl group (e.g., four different alkyl groups). Alternatively, the first R^{a} could be alkyl, the second R^{a} could be fluoro, the first R^{b} could be hydroxyalkyl, and the second R could be amino (or any other substituents taken from the group). Alternatively, both R^{a} and the first R^{b} could be fluoro, while the second R could be alkyl (i.e., some pairs of substituent groups may be the same, while other pairs may be different).

“Amino protecting group” is a protecting group that is suitable for preventing undesired reactions at an amino nitrogen. Representative amino-protecting groups include, but are not limited to, formyl; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBDMS); and the like.

“Hydroxyl protecting group” is a protecting group that is suitable for preventing undesired reactions at a hydroxyl oxygen. Representative hydroxyl protecting groups include, but are not limited to, acyl groups such as acetyl; arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBDMS); ethers such as methoxymethyl (MOM), tetrahydropyranyl (THP), and benzyl (Bn); and the like.

“Yield” for each of the reactions described herein is expressed as a percentage of the theoretical yield.

“Subject and “patient” are used interchangeably. A “subject” or “patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, PA, 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

The phrase “genetic disease”, as used herein, means a genetic disorder, genetic disease, genetic condition or genetic syndrome.

“Preventing” or “prevention” of a disease, disorder, or syndrome includes inhibiting the disease from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

“Treating” or “treatment” of a disease, disorder, or syndrome, as used herein, includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

“FAP” means an autosomal dominant inherited condition in which numerous adenomatous polyps form mainly in the epithelium of the large intestine. While these polyps start out benign, malignant transformation into colon cancer occurs when they are left untreated. Three variants are known to exist: FAP, attenuated FAP (originally called hereditary flat adenoma syndrome), and autosomal recessive FAP (originally called (or MUTYH-associated polyposis). FAP and attenuated FAP are caused by APC gene defects on chromosome 5 while autosomal recessive FAP is caused by defects in the MUTYH gene on chromosome 1. Of the three, FAP itself is the most severe and most common; although for all three, the resulting colonic polyps and cancers are initially confined to the colon wall. Detection and removal before metastasis outside the colon can greatly reduce and in many cases eliminate the spread of cancer.

### Embodiments

In one aspect, what is provided is a method for treating FAP in a patient in need of such treatment, comprising administering to the patient a compound of formula I

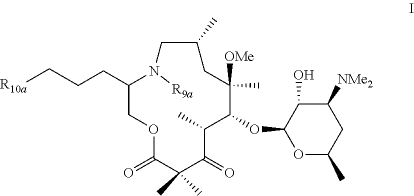

or a pharmaceutically acceptable salt thereof, wherein
R₉ₐ is selected from the group consisting of H or optionally substituted C₁₋₆ alkyl; and
R₁₀ₐ is an optionally substituted heteroaryl.

In one embodiment, R₉ₐ is H

In another embodiment, R₉ₐ is optionally substituted C₁₋₆ alkyl.

In another embodiment, R₉ₐ is selected from optionally substituted methyl, ethyl, propyl, isopropyl, butyl, isobutyl, tert-butyl, pentyl, isopentyl, neopentyl and hexyl.

In another embodiment, R₉ₐ is selected from methyl, ethyl, propyl, butyl, and isobutyl.

In another embodiment, R₁₀ₐ is an optionally substituted heteroaryl selected from the group consisting of

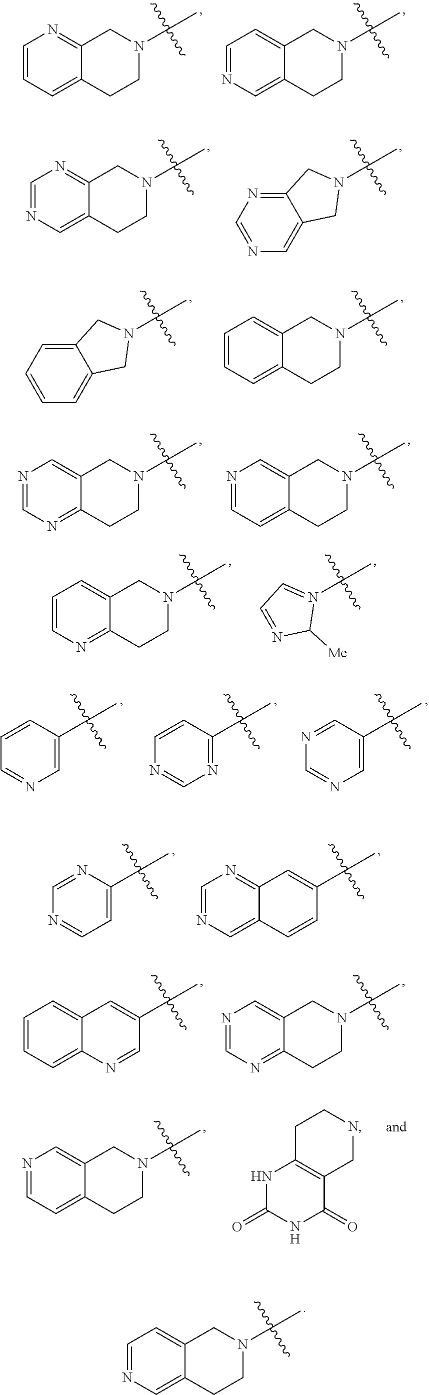

In a further embodiment, R₉ₐ is selected from H, methyl, ethyl, propyl, butyl, and isobutyl and R₁₀ₐ is a bicyclic heteroaryl selected from the group consisting of

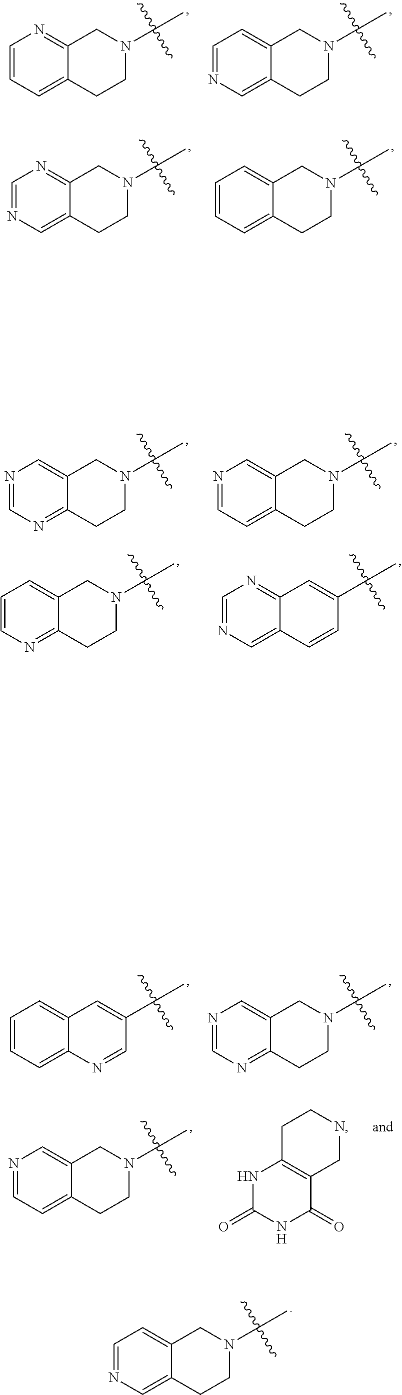

In another aspect, what is provided is a method of treating FAP in a patient in need of such treatment, comprising administering to the patient a compound of formula II

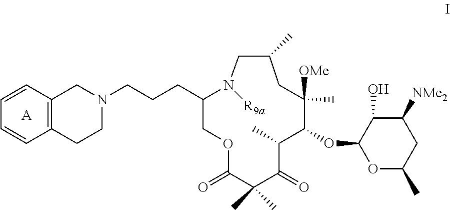

or a pharmaceutically acceptable salt thereof, wherein
R₉ₐ is selected from the group consisting of H or optionally substituted C₁₋₆ alkyl; and

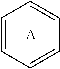

is an optionally substituted 6-membered aromatic or heteroaromatic ring.

In one embodiment, R₉ₐ is H

In another embodiment, R₉ₐ is optionally substituted C₁₋₆ alkyl.

In another embodiment, R₉ₐ is selected from optionally substituted methyl, ethyl, propyl, isopropyl, butyl, isobutyl, tert-butyl, pentyl, isopentyl, neopentyl and hexyl.

In another embodiment, R₉ₐ is selected from methyl, ethyl, propyl, butyl, and isobutyl.

In another embodiment,

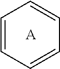

is an optionally substituted aromatic ring.

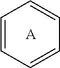

In another embodiment, is an optionally substituted

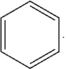

In another embodiment,

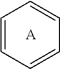

is an optionally substituted heteroaromatic ring.

In another embodiment,

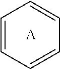

is an optionally substituted

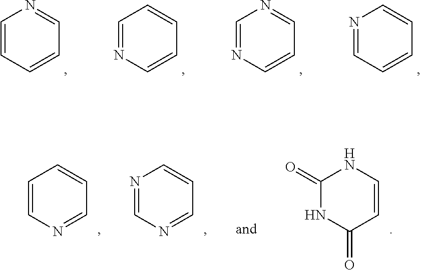

In a further embodiment, R₉ₐ is selected from H, methyl, ethyl, propyl, butyl, and isobutyl and

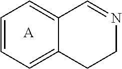

is selected from the group consisting of

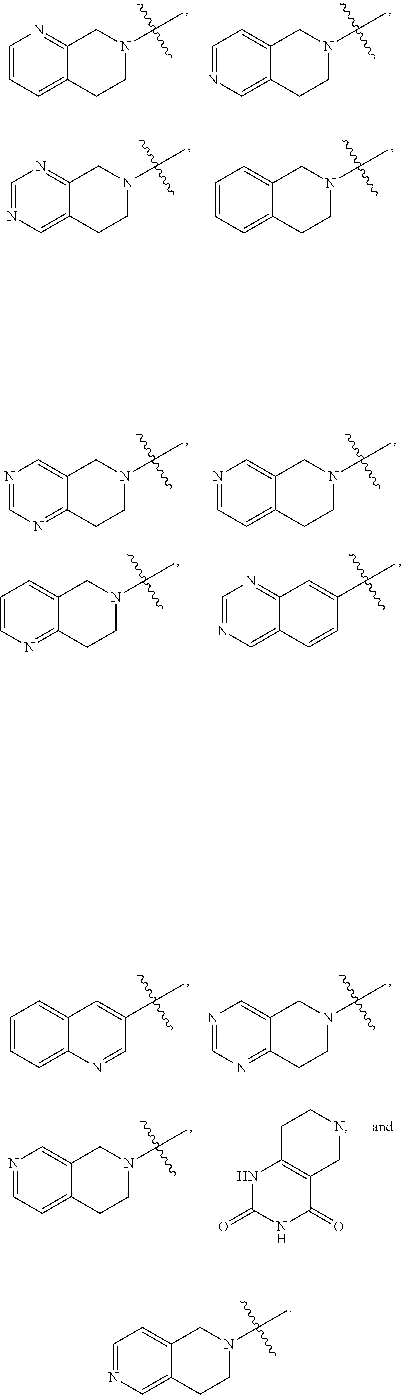

all of which may be optionally substituted.

Compounds of the invention include the compounds depicted in Table A or a pharmaceutically acceptable salt thereof.

TABLE A

| New Cmpd # | Structure |
|---|---|
| 1 | 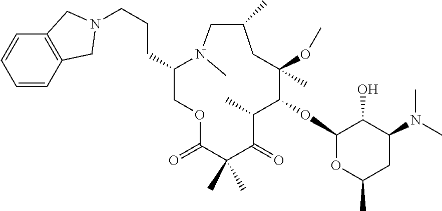 |
| 2 | 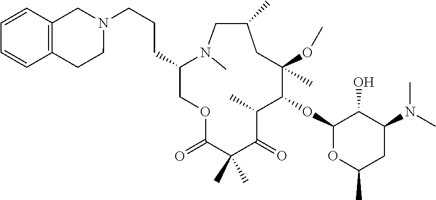 |
| 3 | 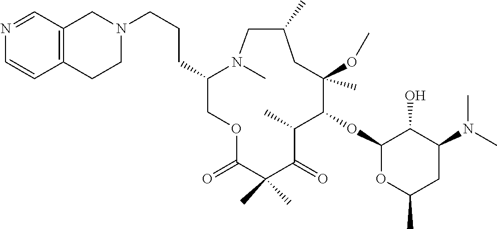 |
| 4 | 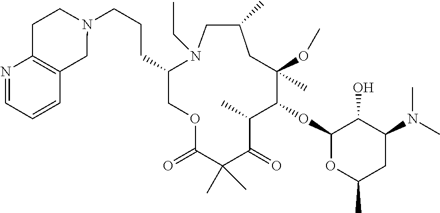 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncccc2C1] |
| 5 | 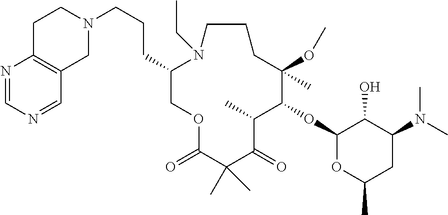 [SMILES: CCN1CCC[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1] |
| 6 | 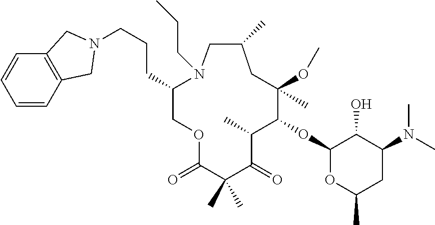 |
| 7 | 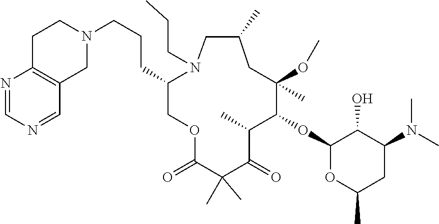 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1] |
| 8 | 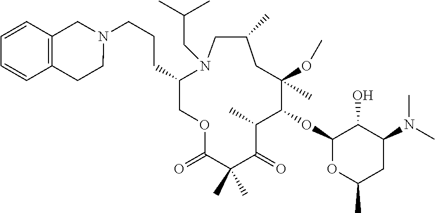 |
| 9 | 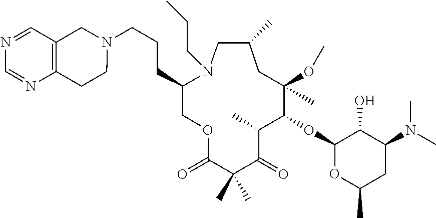 |
| 10 | 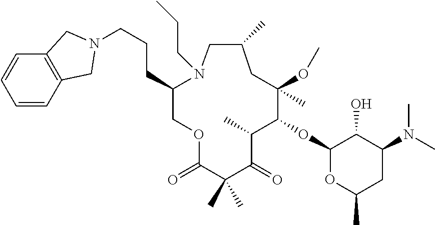 |
| 11 | 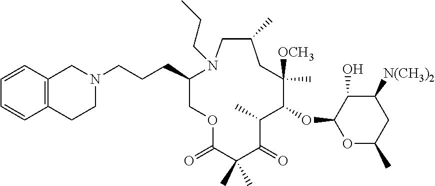 |
| 12 | 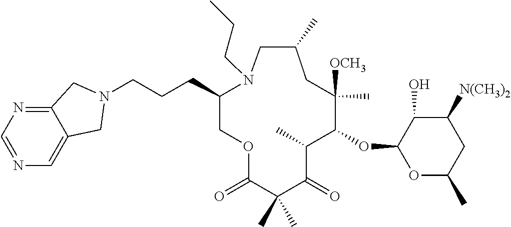 |
| 13 | 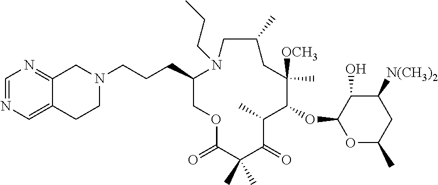 |
| 14 | 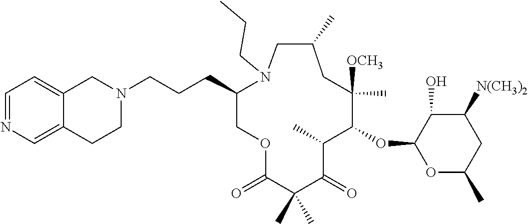 |
| 15 | 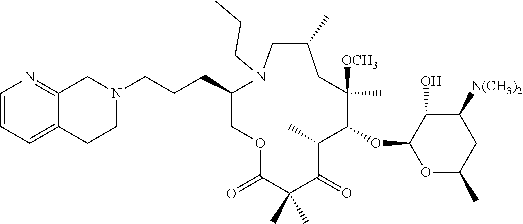 |
| 16 | 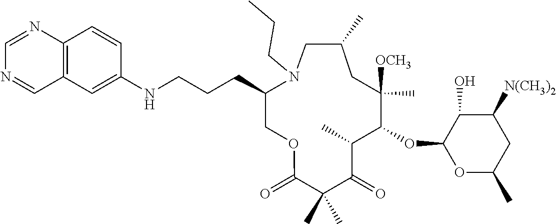 |
| 17 | 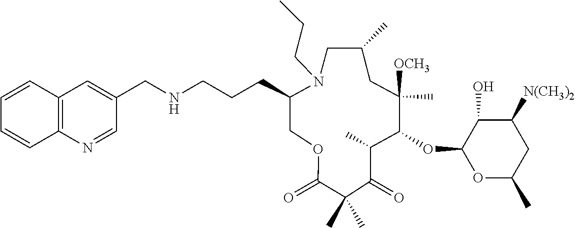 |
| 18 | 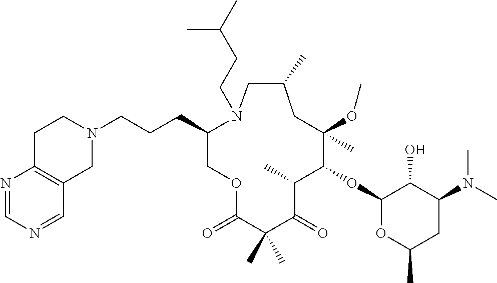 |
| 19 | 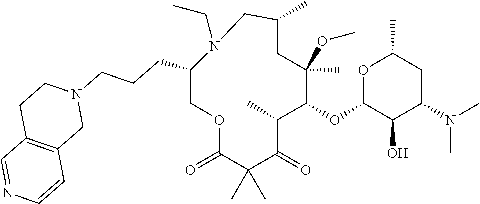 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2cnccc2C1] |
| 20 | 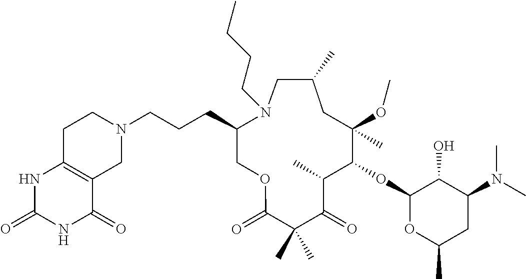 |

### Processes for Preparing Compounds

Compounds disclosed herein can be prepared as provided in WO 2020/106627, the entire contents of which is incorporated herein by reference, or as described herein.

Compounds are prepared via two intermediates. The eastern half intermediate is a compound of formula P-1:

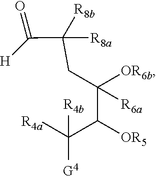

or salt thereof. In the compound of formula P-1, R³, R^{4a}, R^{4b}, R⁵, R^{6a}, R^{6b}, R^{8a}, and R^{8b} are as defined herein; and
G⁴ is of the formula:

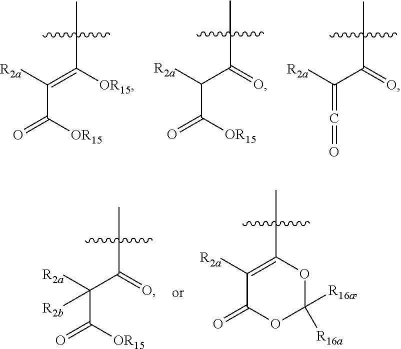

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and
each instance of R^{61a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

The uncyclized eastern half intermediate is a compound of formula P-2:

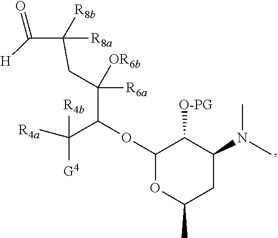

or salt thereof, wherein:
PG is a hydroxyl protecting group;
R₄ₐ, R_{4b}, R₅, R₆ₐ, R_{6b}, R₈ₐ, and R_{8b} are as defined herein;
G⁴ is of formula:

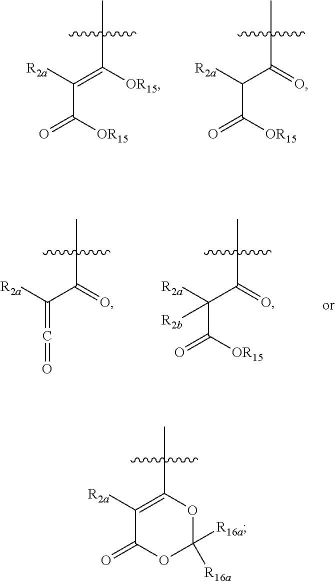

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and
each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

In some embodiments, —OPG is —OBz.

What is also disclosed is a compound of Formula P-3:

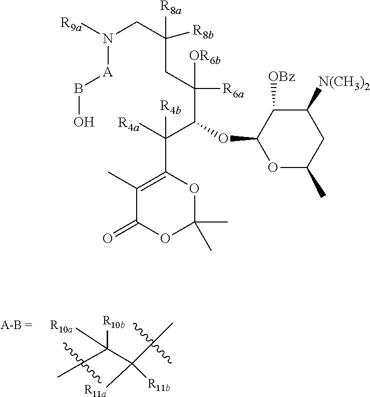

or salt thereof, wherein the variables are as defined herein.

### Coupling and Macrolactonization

In certain embodiments, compounds of the present disclosure are prepared by coupling a compound of Formula P1 (the eastern half) wherein Rₛ is a sugar residue

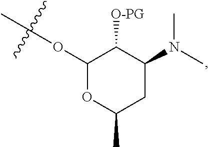

wherein PG is a hydroxyl protecting group and “ [symbol] ” indicates a point of attachment, and a compound of Formula P-4 (the western half) to provide an uncyclized compound precursor of Formula P-5 as depicted in the following Scheme.

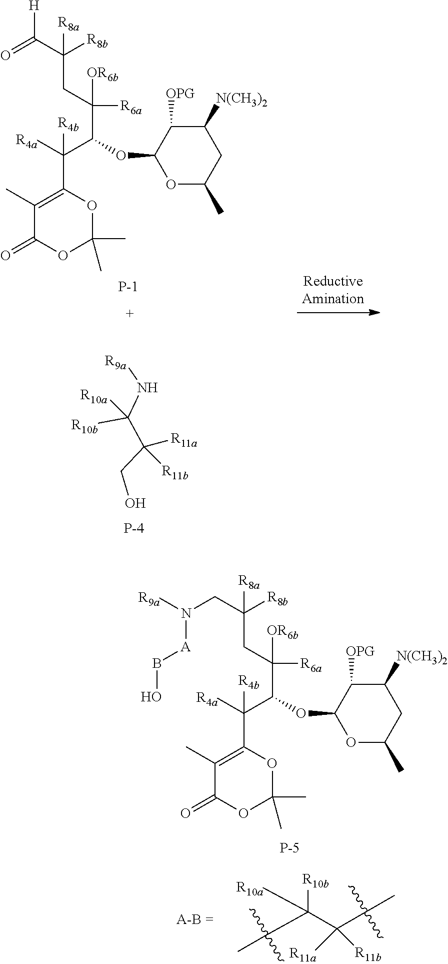

Formula P-5 is cyclized to give, after deprotection of the sugar residue a compound of Formula I as depicted in the following scheme.

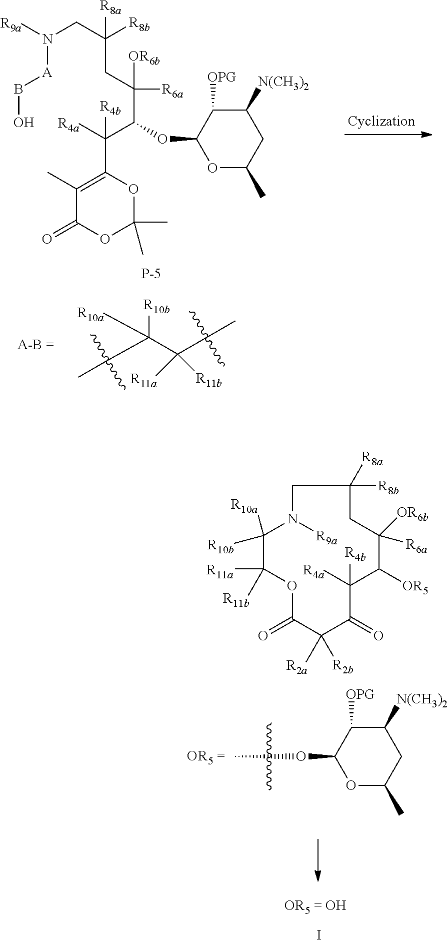

Alternatively, the compound precursor of Formula P-5 wherein R₉ₐ is hydrogen is cyclized to provide a compound of Formula I, which can undergo reductive amination to provide a compound of Formula I wherein R₉ₐ is other than H, as shown in the following Scheme.

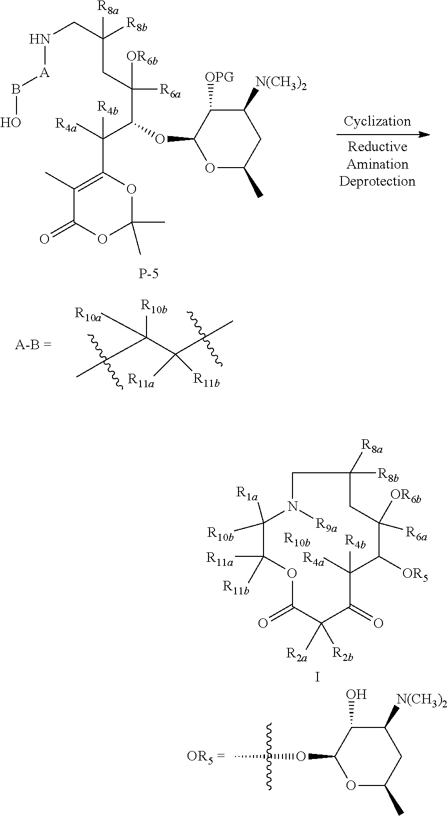

Late-stage installment of the R_{2b} group can be achieved via treatment of a compound of Formula P-6 prepared as provide above with a base and a suitable electrophile group (e.g., halogenating agent or R₂-LG, wherein LG is a leaving group) as depicted in the following Scheme. In this process, the sugar residue in P-6 is protected and R₉ₐ is H or alkyl.

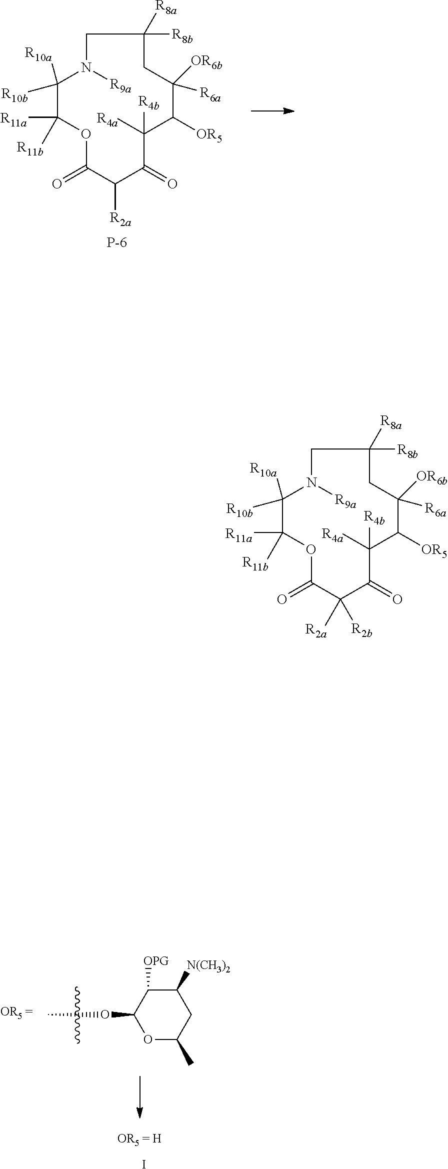

For all intermediates, the variables are as defined herein for a compound of Formula I.

Other variables depicted for intermediates and precursors are defined as follows:
LG is a leaving group;
G⁴ is of formula:

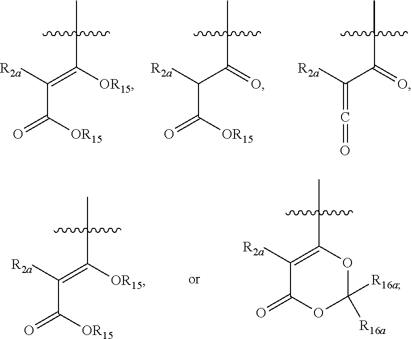

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and
each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

As noted above, Rₛ is the sugar moiety

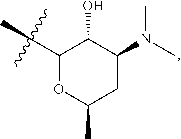 [SMILES: *C1O[C@H](C)C[C@H](N(C)C)[C@H]1O]

The sugar moiety is typically attached to the compound framework during synthesis of the eastern half, but may also be attached at other stages of the preparation. The sugar moiety may be attached by a chemical or enzymatic glycosylation reaction between the hydroxyl group at the C5 position and a glycosyl donor. In certain embodiments, the sugar moiety is attached to the compound framework as a thioglycoside. In certain embodiments, substituents of the sugar moiety are modified after the glycosylation of the compound or compound precursor (e.g., eastern half).

### Methods

In an aspect, the invention provides a method for treating FAP, attenuated FAP (originally called hereditary flat adenoma syndrome), and autosomal recessive FAP (originally called (or MUTYH-associated polyposis), comprising administering to a subject in need of such treatment a therapeutically effective amount of a compound of Formula I.

In one embodiment, the FAP is FAR

In another embodiment, the FAP is attenuated FAP.

In another embodiment, the FAP is autosomal recessive FAP.

In a further aspect, the invention provides a method for treating a genetic disease or genetic disorder caused by APC gene defects on chromosome 5 comprising administering to a subject in need of such treatment a therapeutically effective amount of a compound of Formula I.

In a further aspect, the invention provides a method for treating a genetic disease or genetic disorder caused by a mutation of the APC gene on chromosome 5 comprising administering to a subject in need of such treatment a therapeutically effective amount of a compound of Formula I.

In these and other aspects, the APC mutation is at codon 1309. In another embodiment, the APC mutation is in codons 1250-1464. In a further embodiment, the mutation is between 168 and 1580. In a further embodiment, the mutation is between 5′ of codon 168 and 3′ of 1580.

In these and other aspects, the mutation is in the 5′ part of the APC gene (codons 1-177) and the distal 3′ part of the gene. In a further embodiment, the mutation is an interstitial deletion of chromosome 5q22. In a further embodiment, the mutation is in the 5′end in region spanning exons 4 and 5. In a further embodiment, the mutation is in the 5′end in exon 9.

In a further aspect, the invention provides a method for treating a genetic disease or genetic disorder caused by caused by a mutation of the MUTYH gene on chromosome 1 comprising administering to a subject in need of such treatment a therapeutically effective amount of a compound of Formula I.

In a further aspect, the invention provides a method for treating a genetic disease or genetic disorder caused by caused by gene defects in the MUTYH gene on chromosome 1 comprising administering to a subject in need of such treatment a therapeutically effective amount of a compound of Formula I.

### Pharmaceutical Compositions and Administration

The present invention provides pharmaceutical compositions comprising a compound of the present invention and a pharmaceutically acceptable excipient. In certain embodiments, the compound of the present invention is provided in an effective amount in the pharmaceutical composition. In certain embodiments, the effective amount is a therapeutically effective amount. In certain embodiments, the effective amount is a prophylactically effective amount.

Pharmaceutically acceptable excipients include any and all solvents, diluents, or other liquid vehicles, dispersions, suspension aids, surface active agents, isotonic agents, thickening or emulsifying agents, preservatives, solid binders, lubricants and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the compound of the present invention (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof: malic acid and salts and hydrates thereof: phosphoric acid and salts and hydrates thereof; and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the compounds presented herein may be dissolved or suspended in suitable liquids, such as fatty oils, liquid paraffin, or liquid polyethylene glycols. In addition, stabilizers may be added. All formulations for oral administration should be in dosages suitable for the chosen route of administration.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active aminoglycoside compounds doses.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner Examples of embedding compositions which can be used include polymeric substances and waxes.

Dosage forms for topical and/or transdermal administration of a compound of this invention may include ointments, pastes, creams, lotions, gels, powders, solutions, sprays, inhalants and/or patches. Generally, the active ingredient is admixed under sterile conditions with a pharmaceutically acceptable carrier and/or any needed preservatives and/or buffers as can be required. Additionally, the present invention contemplates the use of transdermal patches, which often have the added advantage of providing controlled delivery of an active ingredient to the body. Such dosage forms can be prepared, for example, by dissolving and/or dispensing the active ingredient in the proper medium. Alternatively, or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537.

Ballistic powder/particle delivery devices which use compressed gas to accelerate vaccine in powder form through the outer layers of the skin to the dermis are suitable. Alternatively, or additionally, conventional syringes can be used in the classical mantoux method of intradermal administration.

A pharmaceutical composition of the invention can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65 °F at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

To practice the method of this invention, the above-described compound or its pharmaceutical composition can be administered intravenously, intravitreally, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference). In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In certain embodiments, the pharmaceutical composition and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally

According to some embodiments, the administration is effected orally. For oral administration, the compounds presented herein can be formulated readily by combining the compounds with pharmaceutically acceptable carriers well known in the art. Such carriers enable the compounds presented herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

For buccal administration, the compositions may take the form of tablets or lozenges formulated in conventional manner.

For administration by inhalation, the compounds presented herein are conveniently delivered in the form of an aerosol spray presentation (which typically includes powdered, liquefied and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compounds presented herein and a suitable powder base such as, but not limited to, lactose or starch.

For administration by injection, the compounds presented herein may be formulated in aqueous solutions, preferably in physiologically compatible buffers such as Hank's solution, Ringer's solution, or physiological saline buffer with or without organic solvents such as propylene glycol, polyethylene glycol.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only.

Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroethylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

For transmucosal administration, penetrants are used in the formulation. Such penetrants are generally known in the art.

The compounds presented herein may be formulated for parenteral administration, e.g., by bolus injection or continuous infusion. Formulations for injection may be presented in unit dosage form, e.g., in ampoules or in multidose containers with optionally, an added preservative.

The compositions may be suspensions, solutions or emulsions in oily or aqueous vehicles, and may contain formulatory agents such as suspending, stabilizing and/or dispersing agents.

Alternatively, the compounds presented herein may be in powder form for constitution with a suitable vehicle, e.g., sterile, pyrogen-free water, before use.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. The compounds or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. Additional therapeutically active agents include antibiotic agents, e.g., antibiotics useful for treating tuberculosis. Exemplary antibiotics include, but are not limited to, isoniazid, rifampin, pyrazinamide, ethambutol, and streptomycin.

Also encompassed by the invention are kits (e.g., pharmaceutical packs). The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

### Examples

In order that the invention described herein may be more fully understood, the following examples are set forth. The synthetic and biological examples described in this application are offered to illustrate the compounds, pharmaceutical compositions, and methods provided herein and are not to be construed in any way as limiting their scope. Compounds were prepared as described in WO 2020/106627 and as described below.

### Intermediate Scheme 6

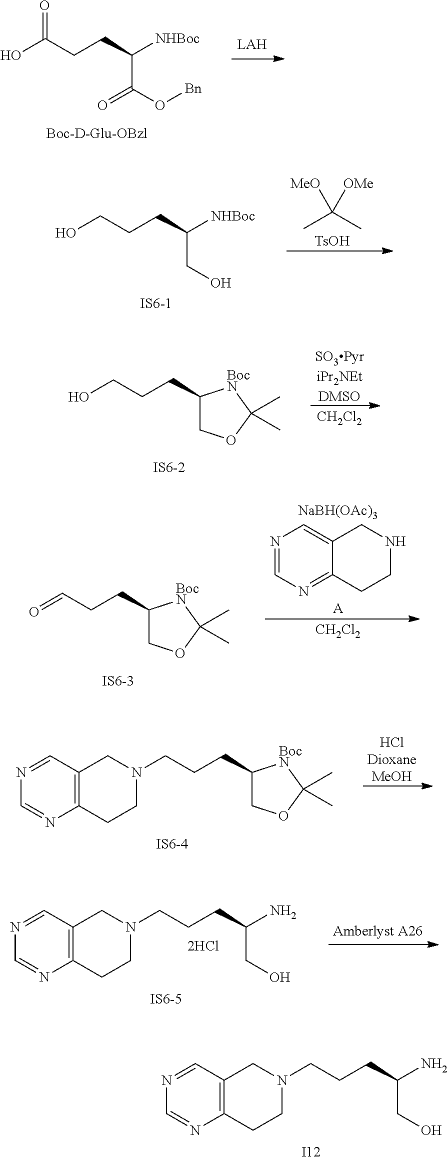

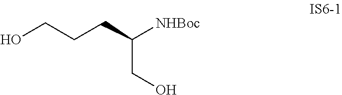 [SMILES: CC(C)(C)OC(=O)N[C@@H](CO)CCCO]

### tert-butyl (R)-(1,5-dihydroxypentan-2-yl)carbamate (IS6-1)

A suspension of LiAlH₄ (1.0 M solution in THF, 800 mL, 800 mmol, 4.2 equiv.) was added drop-wise to a solution of Boc-D-Glu-OBzl (64.1 g, 190 mmol, 1.0 equiv.) in anhydrous tetrahydrofuran (474 mL) at 0 °C. The internal temperature was kept under 10 °C. Upon complete addition, the reaction mixture was allowed to stir at 0 °C for 0.5 h and was then warmed to rt for 1 h. The mixture was cooled to 0 °C and quenched by the cautious addition of water (30.4 mL), 3N NaOH solution (38.4 mL), and then water (84 mL). This mixture was dried with Na₂SO₄, and the precipitate was removed by filtration. The precipitate was washed with EtOAc, and the combined filtrate was concentrated under reduced pressure to give IS6-1. The material was used in the next step without further purification.

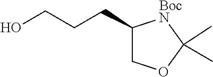

### tert-butyl (R)-4-(3-hydroxypropyl)-2,2-dimethyloxazolidine-3-carboxylate (IS6-2)

A solution of compound IS6-1 (41.6 g, 189 mmol, 1.0 equiv.) in anhydrous methylene chloride (240 mL) was added 2,2-dimethoxypropane (231 mL, 1.89 mol, 10 equiv.) at 25 °C. Then TsOH H₂O (3.59 g, 18.9 mmol, 0.1 equiv.) was added In an portion. The reaction mixture was allowed to stir at 25 °C for 4 h. The mixture was partitioned between EtOAc and sat. aq. NaHCO₃. The organic layer was washed with brine, dried over Na₂SO₄, and concentrated. The mixture was purified by silica gel chromatography (40% EtOAc in Heptane) to give 20.13 g (41% in two steps) compound IS6-2. ¹H NMR (400 MHz, Chloroform-d) δ 4.04-3.91 (m, 2H), 3.79-3.63 (m, 4H), 1.68-1.54 (m, 4H), 1.49 (s, 15H).

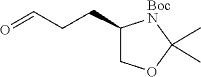

### tert-butyl (R)-2,2-dimethyl-4-(3-oxopropyl)oxazolidine-3-carboxylate (IS6-3)

To a solution of compound IS6-2 (20.13 g, 77.5 mmol, 1.0 equiv) in DCM (155 mL) was added DMSO (44.0 mL, 620 mmol, 8.0 equiv) followed by hunig's base (53.9 mL, 310 mmol, 4.0 equiv) and cooled to 0 °C. To this mixture was added SO₃·Pyr (24.6 g, 155 mmol, 2.0 equiv) in portions maintaining the internal temp below 5 °C. The reaction mixture was allowed to stir at 0-5 °C for 1 h. To the batch was added MTBE and brine (500 mL+500 mL) and stirred for ~10-15 mins. Organic layer was separated and washed with brine (4 times). Final organic layer was dried over sodium sulfate and concentrated to provide crude product IS6-3 and was used in next step without further purification.

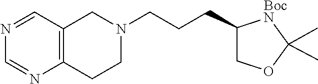

### tert-butyl (R)-4-(3-(7,8-dihydropyrido[4,3-d]pyrimidin-6(5H)-yl)propyl)-2,2-dimethyloxazolidine-3-carboxylate (IS6-4)

To a solution of compound IS6-3 (19.9 g, 77.3 mmol, 1.0 equiv) in DCM (154 mL) was added Compound A (12.5 g, 92.7 mmol, 1.2 equiv) followed by AcOH (4.85 mL, 85.0 mmol, 1.1 equiv) and cooled to 0 °C. To this mixture was added NaBH(OAc)₃ (24.3 g, 115 mmol, 1.5 equiv) in portions maintaining the internal temp below 5 °C. The reaction mixture was allowed to stir at 25 °C for 16 h. Sat. aq. NaHCO₃ was added to the reaction mixture. The organic layer was separated and the aqueous layer was extracted with DCM (3 times). The combined organic layers were dried with sodium sulfate and concentrated in vacuo. The mixture was purified by silica gel chromatography (0-3-5% MeOH in DCM with 0.5% NH₄OH) to gave 28.32 g (97.2% yield) of compound IS6-4.

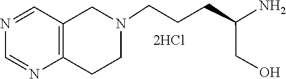 [SMILES: N[C@@H](CO)CCCN1CCc2ncncc2C1]

### (R)-2-amino-5-(7,8-dihydropyrido[4,3-d]pyrimidin-6(5H)-yl)pentan-1-ol hydrochloric acid salt (IS6-5)

Compound IS6-4 (28.32 g, 75.1 mmol, 1.0 equiv.) was dissolved in MeOH (150 mL) and HCl (4 M solution in dioxane, 93.7 mL, 375 mmol, 5.0 equiv.) was added at rt. The reaction mixture was stirred at rt for 4 h, at which point UPLC showed complete conversion. The reaction mixture was concentrated under reduced pressure, yielding 22.9 g (100% crude yield) of Compound IS6-5.

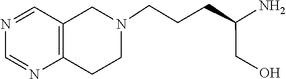 [SMILES: N[C@@H](CO)CCCN1CCc2ncncc2C1]

### (R)-2-amino-5-(7,8-dihydropyrido[4,3-d]pyrimidin-6(5H)-yl)pentan-1-ol (1-12)

Amberlyst A26(OH) (200 g, >0.8 eq/L) was charged with MeOH (500 mL) under mechanical stirrer and the mixture was allowed to stir for 30 min. The solvent was removed by filtration and the same sequence was repeated four times. Compound 5 (22.9 g, 1.0 equiv.) was dissolved in MeOH (500 mL) and was added to the washed resin at rt. The reaction mixture was stirred at rt for 30 min. The solution was collected after filtration and the resin was washed with MeOH (500 mL) three times as before until UPLC shows no desired product was present in the solution. The organic solution was combined and concentrated under reduced pressure, yielding free base (16.97 g, 96.0% yield). ¹H NMR (400 MHz, CDCl₃) δ 8.99 (s, 1H), 8.41 (s, 1H), 3.66-3.57 (m, 3H), 3.51 (s, 2H), 3.32 (dd, 1H), 3.03 (t, 2H), 2.93-2.81 (m, 3H), 2.64-2.54 (m, 2H), 1.80-1.43 (m, 4H).

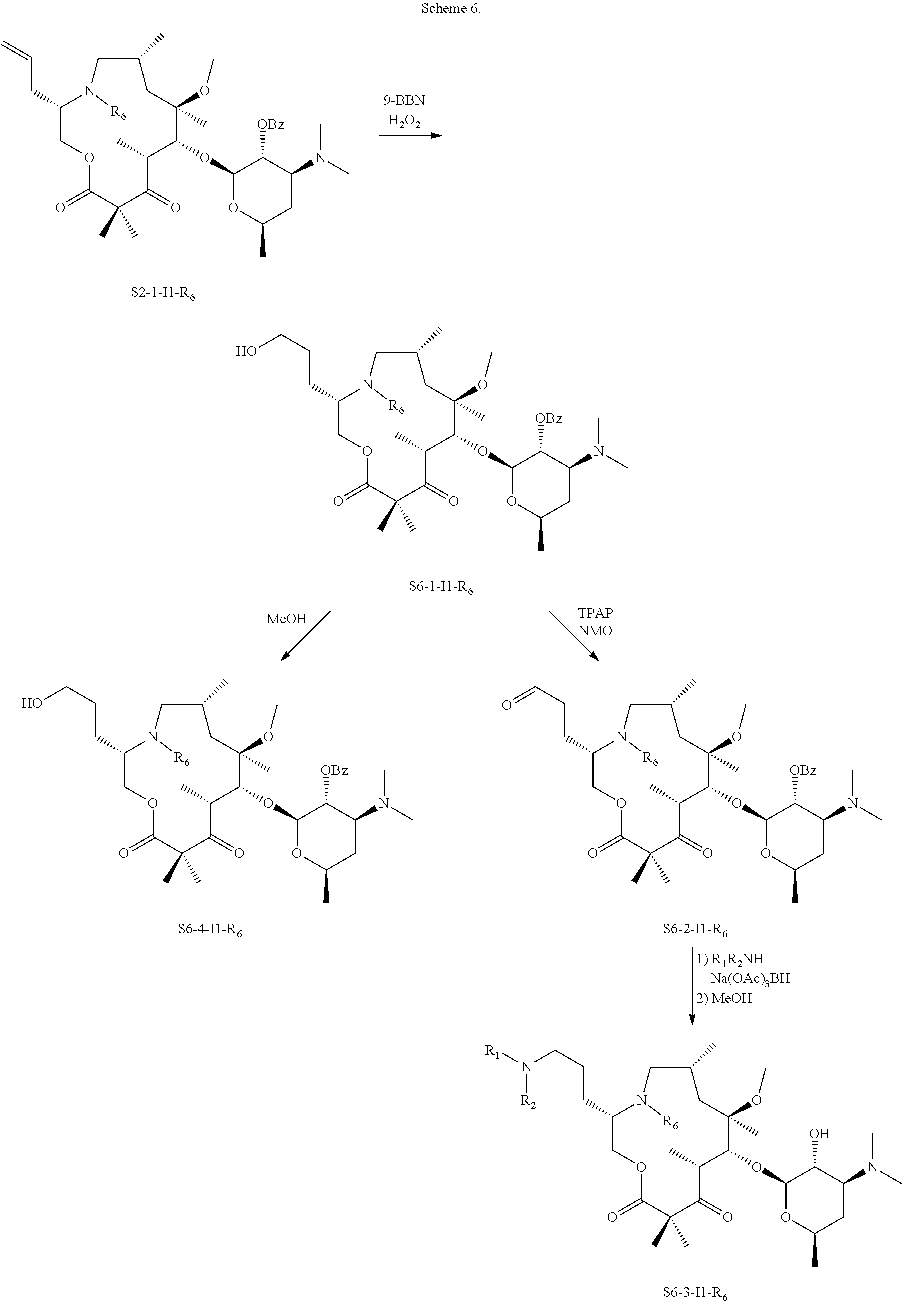

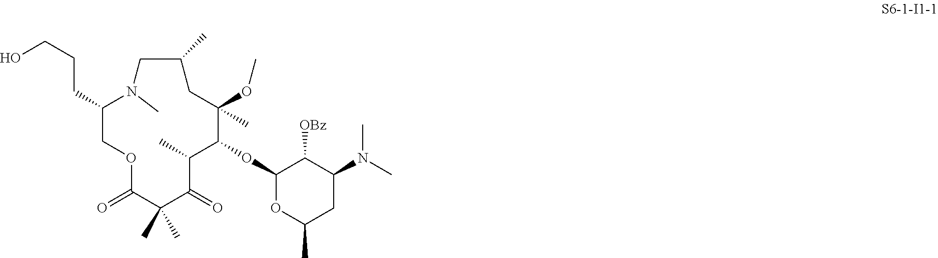

### (2S,3R,4S,6R)-4-(dimethylamino)-2-(((3S,6R,8R,9R,10R)-3-(3-hydroxypropyl)-8-methoxy-4,6,8,10,12,12-hexamethyl-11,13-dioxo-1-oxa-4-azacyclotridecan-9-yl)oxy)-6-methyltetrahydro-2H-pyran-3-yl benzoate (S6-1-I1-1)

To 52-1-I1-1 (240 mg, 0.372 mmol) in dry THF (3.71 mL) was added 9-BBN (0.5M solution in THF, 2.22 mL, 1.11 mmol). After 30 min at rt, the mixture was cooled to 0 °C and NaOH (6 N aqueous solution, 371 μL, 2.23 mmol) and H₂O₂ (30% aqueous solution, 252 μL, 2.23 mmol) were added. After 15 min, the mixture was extracted with t-butylmethylether/EtOAc (2:1) three times. The organic layer was washed with water (1 time) and brine (1 time) and was dried over Na₂SO₄. After the solvent was removed, the residue was purified on 4 g of silica gel (elution with 0-20% MeOH— dichloromethane/0.5% NH₄OH gradient) to give the title compound (145 mg, 59%). MS (ESI+) m/z: 663.37 [M+H]+; 1H NMR (400 MHz, Chloroform-d) δ 8.08-7.94 (m, 2H), 7.55 (dd, 1H), 7.44 (t, 2H), 5.03 (dd, 1H), 4.57 (d, 1H), 4.10 (dd, 1H), 4.01 (d, 1H), 3.95 (dd, 1H), 3.72-3.50 (m, 3H), 3.41 (dt, 1H), 3.04 (s, 1H), 2.87-2.81 (m, 1H), 2.80 (s, 3H), 2.32 (dd, 1H), 2.26 (s, 6H), 2.10 (t, 1H), 1.93 (d, 1H), 1.83-1.47 (m, 10H), 1.40 (s, 4H), 1.31-1.22 (m, 9H), 1.16-1.07 (m, 1H), 1.03 (d, 3H), 0.91 (d, 3H).

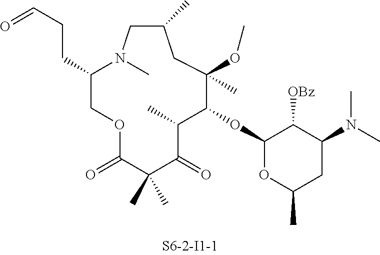

### (2S,3R,4S,6R)-4-(dimethylamino)-2-(((3S,6R,8R,9R,10R)-8-methoxy-4,6,8,10,12,12-hexamethyl-11,13-dioxo-3-(3-oxopropyl)-1-oxa-4-azacyclotridecan-9-yl)oxy)-6-methyltetrahydro-2H-pyran-3-yl benzoate (S6-2-I1-1)

To 56-1-I1-1 (145 mg, 218 mmol) in dry dichloromethane/CH3CN (9:1, 2.9 mL) was added activated 4 A molecular sieves (100 mg, powdered), N-methylmorpholine N-oxide (33 mg, 283 mmol), and tetrapropylammonium perruthenate (4 mg, 10.9 mmol). After 1 h at RT, the solvent was removed. The dried residue was dissolved in t-butylmethylether/Hexane (1:1) and was filtered through Celite® (3 times). After the solvent was removed, the residue was dried under vacuum to give the aldehyde as a white foam. MS (ESI+) m/z: 661.35 [M+H]+. Used directly in the next step.

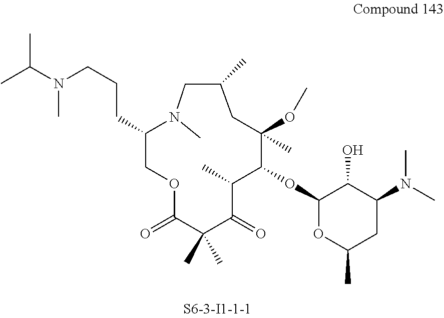

### (3S,6R,8R,9R,10R)-9-(((2S,3R,4S,6R)-4-(dimethylamino)-3-hydroxy-6-methyltetrahydro-2H-pyran-2-yl)oxy)-3-(3-(isopropyl(methyl)amino)propyl)-8-methoxy-4,6,8,10,12,12-hexamethyl-1-oxa-4-azacyclotridecane-11,13-dione (56-3-I1-1-1)

A mixture of S6-2-I1-1 (25 mg, 37.8 mmol) and methylisopropyamine (8 mg, 113 mmol) in dichloromethane (2 mL) was stirred for 30 min, then NaBH(OAc)3 (12 mg, 56.7 mmol) was added. After 20 min, the solvent was removed, and the residue was dissolved in MeOH (2 mL) and was heated at 50 °C overnight. The reaction was allowed to cool to rt and was concentrated. The residue was purified by HPLC (Atlantis T3 column, 5-50% MeCN-water-0.1% HCO2H) to give 8.6 mg of the title compound as a formate salt. MS (ESI+) m/z: 614.48 [M+H]+; 1H NMR (400 MHz, Methanol-d4) δ 4.45 (d, 1H), 4.28 (d, 1H), 4.20 (d, 1H), 3.88-3.65 (m, 2H), 3.56 (hept, 1H), 3.51-3.23 (m, 4H), 3.05 (t, 7H), 2.79 (s, 8H), 2.72 (s, 3H), 2.17 (s, 1H), 2.02 (ddd, 1H), 1.84 (d, 4H), 1.67-1.41 (m, 7H), 1.45-1.19 (m, 19H), 1.05 (d, 3H).

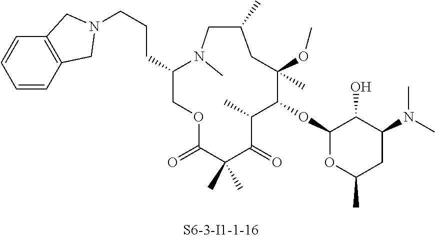

### (3S,6R,8R,9R,10R)-9-(((2S,3R,4S,6R)-4-(dimethylamino)-3-hydroxy-6-methyltetrahydro-2H-pyran-2-yl)oxy)-3-(3-(isoindolin-2-yl)propyl)-8-methoxy-4,6,8,10,12,12-hexamethyl-1-oxa-4-azacyclotridecane-11,13-dione (56-3-I1-1-16)

Prepared according to the methods of S6-3-I1-1-1 from isoindoline to give the title compound as a formate salt. (ESI+) m/z: 660.29 [M+H]+; 1H NMR (400 MHz, Methanol-d4) δ 7.27 (p, 4H), 4.46 (d, 1H), 4.26 (dd, 2H), 4.11 (s, 4H), 3.89-3.64 (m, 2H), 3.53-3.27 (m, 5H), 3.18-2.91 (m, 9H), 2.80 (s, 7H), 2.20 (s, 1H), 2.10-1.92 (m, 2H), 1.75 (ddd, 3H), 1.47 (d, 6H), 1.44-1.29 (m, 12H), 1.06 (d, 3H).

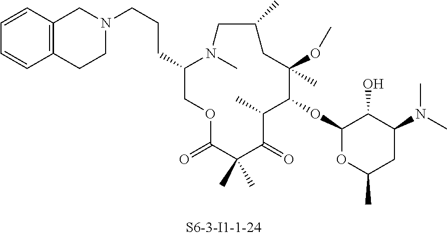

### (3S,6R,8R,9R,10R)-3-(3-(3,4-dihydroisoquinolin-2(1H)-yl)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-24)

Prepared according to the methods of S6-3-I1-1-1 from 1,2,3,4-tetrahydroisoquinoline to give the title compound as a formate salt. MS (ESI+) m/z: 674.33 [M+H]+; 1H NMR (400 MHz, Methanol-d4) δ 7.28-6.98 (m, 4H), 4.45 (d, 1H), 4.27 (dd, 2H), 3.98-3.62 (m, 4H), 3.55-3.34 (m, 4H), 3.19-2.85 (m, 11H), 2.77 (s, 10H), 2.18 (d, 1H), 2.01 (ddd, 2H), 1.91-1.65 (m, 3H), 1.65-1.44 (m, 6H), 1.44-1.25 (m, 12H), 1.05 (d, 3H).

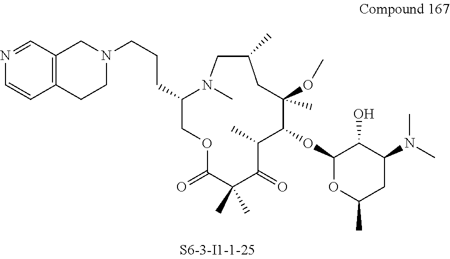

### (3S,6R,8R,9R,10R)-3-(3-(3,4-dihydro-2,7-naphthyridin-2(H)-yl)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-25)

Prepared according to the methods of S6-3-I1-1-1 from 1,2,3,4-tetrahydro-2,7-naphthyridine to give the title compound as a formate salt. MS (ESI+) m/z: 675.28 [M+H]+; 1H NMR (400 MHz, Methanol-d4) δ 8.32 (s, 1H), 8.26 (d, 1H), 7.16 (s, 1H), 4.45 (d, 1H), 4.27 (dd, 2H), 3.89-3.64 (m, 4H), 3.52-3.32 (m, 4H), 2.99 (dd, 9H), 2.89-2.75 (m, 9H), 2.66 (t, 2H), 2.20 (s, 1H), 2.03 (ddd, 2H), 1.89-1.64 (m, 3H), 1.63-1.45 (m, 6H), 1.45-1.26 (m, 12H), 1.05 (d, 3H).

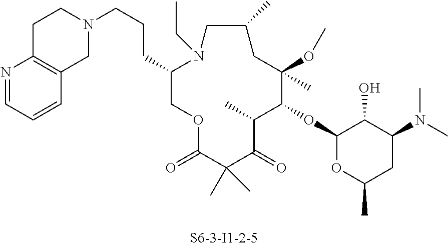 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncccc2C1]

### (3S,6R,8R,9R,10R)-3-(3-(7,8-dihydro-1,6-naphthyridin-6(5H)-yl)propyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S6-3-I1-2-5)

Prepared according to the methods of S6-3-I1-1-1 from S2-1-I1-2 and 5,6,7,8-tetrahydro-1,6-naphthyridine to give the title compound as a formate salt. MS (ESI+) m/z: 689.36 [M+H]+; 1H NMR (400 MHz, MeOD-d4): δ 8.42 (s, 3H), 8.33 (dd, 1H), 7.67-7.46 (m, 1H), 7.30-7.06 (m, 1H), 4.59 (t, 1H), 4.51-4.37 (m, 2H), 4.25 (d, 1H), 4.00 (s, 1H), 3.77 (s, 2H), 3.75-3.65 (m, 1H), 3.55 (s, 1H), 3.47-3.32 (m, 3H), 3.12 (d, 2H), 2.99 (td, 7H), 2.89-2.82 (m, 1H), 2.81 (d, 6H), 2.76-2.66 (m, 2H), 2.14 (s, 1H), 2.07-1.98 (m, 1H), 1.94 (s, 1H), 1.87-1.69 (m, 3H), 1.56-1.47 (m, 1H), 1.44 (s, 3H), 1.40-1.23 (m, 16H), 1.04 (d, 3H).

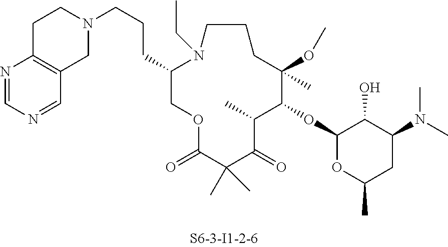 [SMILES: CCN1CCC[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1]

### (3S,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-4-ethyl-8-methoxy-8,10,12,12-tetramethyl-1-oxa-4-azacyclotridecane-11,13-dione (S6-3-I1-2-6)

Prepared according to the methods of S6-3-I1-1-1 from S2-1-I1-2 and 5,6,7,8-tetrahydropyrido[4,3-d]pyrimidine to give the title compound as a formate salt. MS (ESI+) m/z: 690.33 [M+H]+; 1H NMR (400 MHz, MeOD-d4): δ 8.90 (s, 1H), 8.51 (s, 1H), 8.46 (s, 2H), 4.60 (t, 1H), 4.45 (dd, 2H), 4.26 (d, 1H), 3.99 (s, 1H), 3.71 (s, 3H), 3.55 (s, 1H), 3.50-3.33 (m, 3H), 3.25-3.06 (m, 2H), 3.00 (d, 5H), 2.95-2.83 (m, 3H), 2.81 (s, 6H), 2.68 (tt, 2H), 2.18 (d, 1H), 2.08-1.99 (m, 1H), 1.95 (s, 1H), 1.88-1.68 (m, 3H), 1.58-1.49 (m, 1H), 1.45 (s, 3H), 1.42-1.20 (m, 16H), 1.04 (d, 3H).

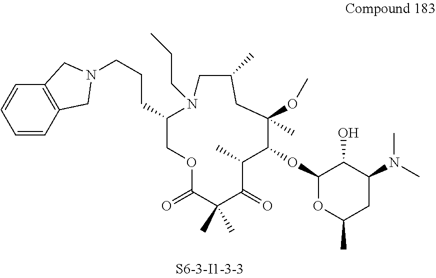

### (3S,6R,8R,9R,10R)-9-(((2S,3R,4S,6R)-4-(dimethylamino)-3-hydroxy-6-methyltetrahydro-2H-pyran-2-yl)oxy)-3-(3-(isoindolin-2-yl)propyl)-8-methoxy-6,8,10,12,12-pentamethyl-4-propyl-1-oxa-4-azacyclotridecane-11,13-dione (S6-3-I1-3-3)

Prepared according to the methods of S6-3-I1-1-1 from S2-1-I1-3 and isoindoline to give 11.9 mg of the title compound as a formate salt. MS (ESI+) m/z: 230.1 [M+3H]3+, 344.7 [M+2H]2+, 688.3 [M+H]+; 1H NMR (400 MHz, Methanol-d4) δ 8.54 (s, 2H), 7.26 (s, 4H), 4.69-4.20 (m, 3H), 4.20-3.86 (m, 6H), 3.69 (dd, 1H), 3.65-3.51 (m, 1H), 3.48-3.34 (m, 2H), 3.28-3.09 (m, 2H), 3.00 (s, 2H), 2.96-2.78 (m, 4H), 2.78-2.51 (m, 7H), 2.51-2.10 (m, 2H), 2.00-1.89 (m, 2H), 1.87-1.58 (m, 5H), 1.58-1.19 (m, 19H), 1.14-0.82 (m, 7H).

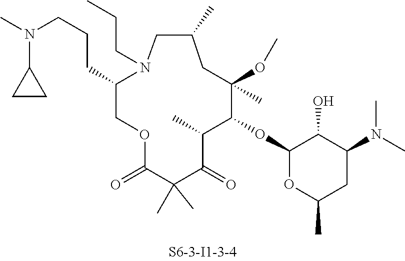

### (3S,8R,9R,10R)-3-(3-(cyclopropyl(methyl)amino)propyl)-9-(((2S,3R,4S,6R)-4-(dimethylamino)-3-hydroxy-6-methyltetrahydro-2H-pyran-2-yl)oxy)-8-methoxy-8,10,12,12-tetramethyl-4-propyl-1-oxa-4-azacyclotridecane-11,13-dione (S6-3-I1-3-4)

Prepared according to the methods of S6-3-I1-1-1 from S2-1-I1-3 and N-methylcyclopropaneamine to give the title compound as a formate salt. MS (ESI+) m/z: 640.03 [M+H]+; ¹H NMR (400 MHz, Methanol-d₄): δ 4.57 (s, 1H), 4.42 (dd, 2H), 4.25 (s, 1H), 3.90 (s, 1H), 3.78-3.64 (m, 1H), 3.53-3.32 (m, 3H), 3.22 (d, 2H), 3.08-2.91 (m, 3H), 2.81 (s, 9H), 2.53 (s, 4H), 2.18 (s, 1H), 2.11-1.88 (m, 3H), 1.73 (s, 4H), 1.61-1.20 (m, 19H), 1.06 (t, 6H), 0.78-0.46 (m, 4H).

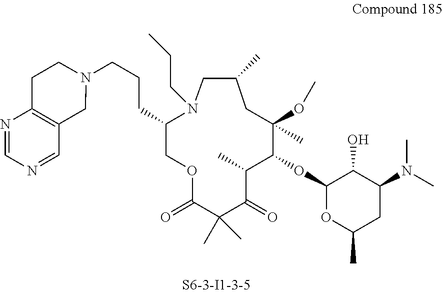 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1]

### (3S,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I1-3-5)

Prepared according to the methods of 56-3-I1-1-1 from 52-1-I1-3 and 5,6,7,8-tetrahydropyrido[4,3-d]pyrimidine to give the title compound as a formate salt. MS (ESI+) m/z: 703.98 [M+H]+; ¹H NMR (400 MHz, Methanol-d₄): δ 8.91 (s, 1H), 8.52 (s, 1H), 4.57 (d, 1H), 4.46 (dd, 2H), 4.25 (s, 1H), 3.98 (s, 1H), 3.81-3.64 (m, 3H), 3.51-3.33 (m, 3H), 3.17 (s, 2H), 3.01 (d, 5H), 2.94-2.85 (m, 3H), 2.82 (s, 7H), 2.69 (hept, 2H), 2.18 (s, 1H), 2.09-1.89 (m, 3H), 1.76 (ddt, 4H), 1.57-1.25 (m, 19H), 1.09-0.92 (m, 6H).

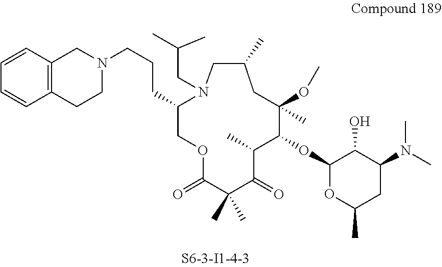

### (3S,6R,8R,9R,10R)-3-(3-(3,4-dihydroisoquinolin-2(1H)-yl)propyl)-9-(((2S,3R,4S,6R)-4-(dimethylamino)-3-hydroxy-6-methyltetrahydro-2H-pyran-2-yl)oxy)-4-isobutyl-8-methoxy-6,8,10,12,12-pentamethyl-1-oxa-4-azacyclotridecane-11,13-dione (S6-3-I1-4-3)

Prepared according to the methods of 56-3-I1-1-1 from 52-1-I1-4 and 1,2,3,4-tetrahydroisoquinoline to give the title compound as a formate salt. MS (ESI+) m/z: 716.32 [M+H]+; 1H NMR (400 MHz, Methanol-d4) δ 7.41-6.89 (m, 4H), 4.66-4.40 (m, 2H), 4.27-4.01 (m, 3H), 4.01-3.62 (m, 3H), 3.62-3.30 (m, 3H), 3.15 (dd, 5H), 3.05-2.67 (m, 11H), 2.66-2.01 (m, 6H), 2.01-1.49 (m, 10H), 1.49-1.25 (m, 12H), 1.25-1.02 (m, 5H), 1.02-0.77 (m, 5H).

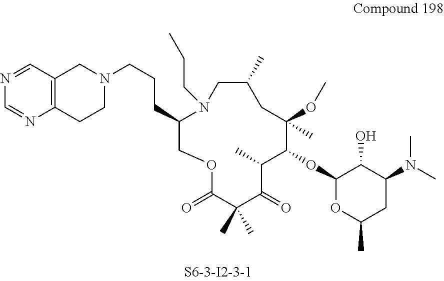

### (3R,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-12-3-1)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 5,6,7,8-tetrahydropyrido[4,3-d]pyrimidine to give the title compound as a formate salt. MS (ESI+) m/z: 704.04 [M+H]+; 1H NMR (400 MHz, Methanol-d4) δ 8.93 (s, 1H), 8.54 (s, 1H), 4.45 (d, 1H), 4.41-4.10 (m, 2H), 3.73 (q, 4H), 3.57-3.16 (m, 6H), 3.08-2.87 (m, 9H), 2.84 (s, 6H), 2.80-2.63 (m, 2H), 2.18-1.60 (m, 8H), 1.61-1.44 (m, 6H), 1.42-1.22 (m, 13H), 1.01-0.85 (m, 6H).

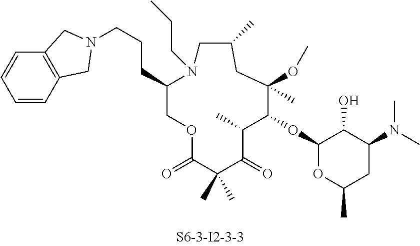

### (3R,6R,8R,9R,10R)-9-(((2S,3R,4S,6R)-4-(dimethylamino)-3-hydroxy-6-methyltetrahydro-2H-pyran-2-yl)oxy)-3-(3-(isoindolin-2-yl)propyl)-8-methoxy-6,8,10,12,12-pentamethyl-4-propyl-1-oxa-4-azacyclotridecane-11,13-dione (S6-3-12-3-3)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and isoindoline to give the title compound as a formate salt. MS (ESI+) m/z: 688.27 [M+H]+; 1H NMR (400 MHz, Methanol-d4) δ 7.29 (t, 4H), 4.46 (d, 1H), 4.18 (s, 6H), 3.72 (ddt, 1H), 3.61-3.31 (m, 5H), 2.98 (d, 8H), 2.79 (s, 7H), 2.02 (ddd, 2H), 1.82 (s, 7H), 1.51 (d, 5H), 1.44-1.23 (m, 13H), 1.00 (t, 6H).

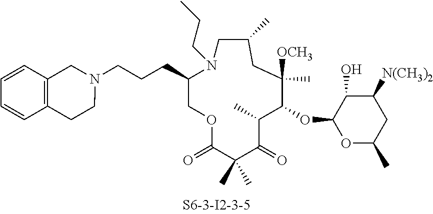

### (3R,6R,8R,9R,10R)-3-(3-(3,4-Dihydroisoquinolin-2(1H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-5)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 1,2,3,4-tetrahydroisoquinoline to give the title compound as a formate salt. MS (ESI+) m/z: 234.8 [M+3H]3+, 351.8 [M+2H]2+, 702.5 [M+H]+; ¹H NMR (400 MHz, Methanol-d₄) δ 6.91-6.82 (m, 1H), 6.76 (dd, 1H), 6.48 (d, 1H), 6.38 (td, 1H), 4.25 (d, 1H), 4.08 (dd, 1H), 3.87 (d, 1H), 3.77 (t, 1H), 3.55 (ddt, 1H), 3.46 (dtt, 1H), 3.16 (dt, 5H), 2.88 (d, 1H), 2.65 (d, 3H), 2.62 (s, 1H), 2.62-2.41 (m, 3H), 2.24 (s, 6H), 2.17 (d, 1H), 1.95 (dd, 1H), 1.88-1.78 (m, 2H), 1.65 (ddd, 1H), 1.55 (s, 1H), 1.54-1.47 (m, 3H), 1.41 (s, 3H), 1.39-1.31 (m, 1H), 1.23 (s, 3H), 1.21-1.09 (m, 10H), 0.93-0.77 (m, 4H), 0.72 (d, 3H).

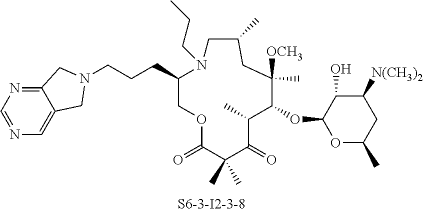

### (3R,6R,8R,9R,10R)-3-(3-(5,7-Dihydro-6H-pyrrolo[3,4-d]pyrimidin-6-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-12-3-8)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 6,7-dihydro-5H-pyrrolo[3,4-d]pyrimidine to give the title compound as a formate salt. MS (ESI+) m/z: 230.8 [M+3H]3+, 345.8 [M+2H]2+, 690.5 [M+H]+; ¹H NMR (400 MHz, Methanol-d₄) δ 9.03 (s, 1H), 8.66 (s, 1H), 8.50 (s, 2H), 4.46 (d, 1H), 4.21 (dd, 2H), 4.06-3.98 (m, 3H), 3.74 (ddt, 1H), 3.51 (dd, 1H), 3.42 (ddd, 2H), 3.00 (s, 2H), 2.95 (t, 2H), 2.84 (s, 3H), 2.10-2.01 (m, 1H), 1.61-1.47 (m, 5H), 1.44-1.30 (m, 12H), 1.02 (t, 5H).

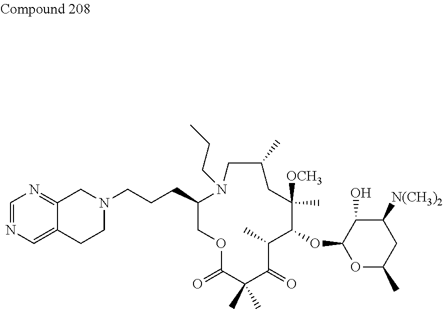

### (3R,6R,8R,9R,10R)-3-(3-(5,8-dihydropyrido[3,4-d]pyrimidin-7(6H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (56-3-12-3-11)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 5,6,7,8-tetrahydropyrido[3,4-d]pyrimidine to give the title compound as a formate salt. MS (ESI+) m/z: 235.5 [M+3H]3+, 352.8 [M+2H]2+, 704.5 [M+H]+; ¹H NMR (400 MHz, Methanol-d4) δ 8.93 (s, 1H), 8.61 (s, 1H), 8.47 (s, 2H), 4.46 (d, 1H), 4.35 (s, 1H), 4.25 (d, 1H), 3.74 (s, 3H), 3.51 (dd, 1H), 3.42 (ddd, 1H), 3.29 (s, 2H), 3.00 (d, 5H), 2.92 (q, 1H), 2.85 (s, 7H), 2.74 (qd, 2H), 2.06 (ddd, 1H), 1.96 (s, 1H), 1.87-1.77 (m, 2H), 1.77-1.72 (m, 1H), 1.61-1.55 (m, 1H), 1.53 (s, 4H), 1.41-1.28 (m, 13H), 1.04-0.96 (m, 6H).

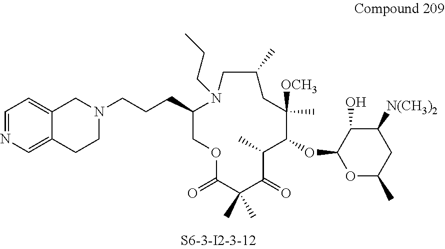

### (3R,6R,8R,9R,10R)-3-(3-(3,4-dihydro-2,6-naphthyridin-2(1H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-12)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 1,2,3,4-tetrahydro-2,6-naphthyridine to give the title compound as a formate salt. MS (ESI+) m/z: 235.2 [M+3H]3+, 352.3 [M+2H]2+, 703.5 [M+H]+; ¹H NMR (400 MHz, Methanol-d₄) δ 8.48 (s, 2H), 8.36-8.17 (m, 2H), 7.26 (d, 1H), 4.46 (d, 1H), 4.35 (s, 1H), 4.25 (d, 1H), 3.87-3.66 (m, 4H), 3.55-3.38 (m, 2H), 3.28 (s, 1H), 3.01 (s, 6H), 2.85 (s, 6H), 2.06 (ddd, 2H), 1.95 (s, 11H), 1.75 (s, 1H), 1.54 (s, 3H), 1.37 (dt, 14H), 1.00 (t, 3H), 0.91 (d, 3H).

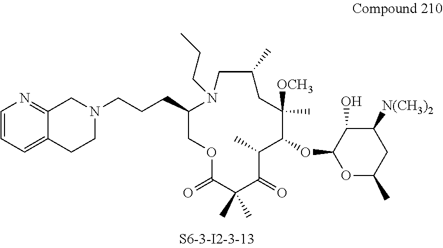

### (3R,6R,8R,9R,10R)-3-(3-(5,8-Dihydro-1,7-naphthyridin-7(6H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (56-3-12-3-13)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 5,6,7,8-tetrahydro-1,7-naphthyridine to give the title compound as a formate salt. MS (ESI+) m/z: 235.2 [M+3H]3+, 352.3 [M+2H]2+, 703.5 [M+H]+; ¹H NMR (400 MHz, Methanol-d₄) δ 8.46 (s, 2H), 8.36 (dd, 1H), 7.62 (dd, 1H), 7.27 (dd, 1H), 4.46 (d, 1H), 4.36 (s, 1H), 4.27 (d, 1H), 3.86-3.75 (m, 3H), 3.75-3.71 (m, 1H), 3.55-3.39 (m, 2H), 3.11-2.97 (m, 8H), 2.92 (d, 1H), 2.85 (s, 6H), 2.77 (t, 2H), 2.06 (dt, 1H), 1.95 (s, 1H), 1.84 (dt, 2H), 1.77 (s, 1H), 1.62-1.49 (m, 6H), 1.42-1.31 (m, 13H), 1.00 (t, 3H), 0.92 (d, 3H).

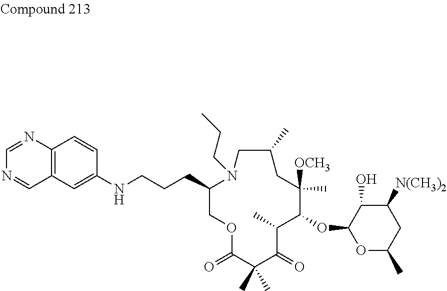

### (3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-4-propyl-3-(3-(quinazolin-6-ylamino)propyl)-1-oxa-4-azacyclotridecane-11,13-dione (S6-3-12-3-16)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and quinazolin-6-amine to give the title compound as a formate salt. MS (ESI+) m/z: 238.8 [M+3H]3+, 357.8 [M+2H]2+, 714.5 [M+H]+; ¹H NMR (400 MHz, Methanol-d₄) δ 9.09 (s, 1H), 8.75 (s, 1H), 8.45 (s, 1H), 7.64 (d, 11H), 7.39 (dd, 1H), 6.78 (d, 1H), 4.30 (d, 1H), 4.18 (d, 1H), 3.86 (d, 1H), 3.78 (t, 1H), 3.52 (ddt, 1H), 3.30-3.23 (m, 2H), 3.17 (s, 1H), 2.98 (s, 1H), 2.86 (s, 1H), 2.67 (s, 2H), 2.45 (s, 4H), 2.42 (s, 3H), 1.86 (t, 1H), 1.76 (d, 1H), 1.64 (s, 2H), 1.50 (s, 1H), 1.41 (s, 3H), 1.37-1.25 (m, 4H), 1.25-1.10 (m, 13H), 0.84-0.66 (m, 4H), 0.60 (d, 2H).

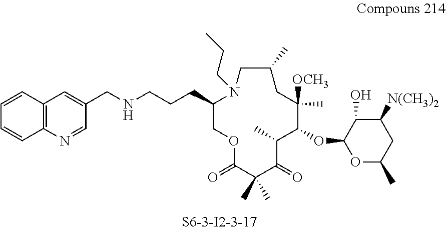

### (3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-4-propyl-3-(3-((quinolin-3-ylmethyl)amino)propyl)-1-oxa-4-azacyclotridecane-11,13-dione (S6-3-I2-3-17)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and quinolin-3-ylmethanamine to give the title compound as a formate salt. MS (ESI+) m/z: 243.2 [M+3H]3+, 364.3 [M+2H]2+, 727.5 [M+H]+; ¹H NMR (400 MHz, Methanol-d₄) δ 8.95 (d, 1H), 8.53 (s, 2H), 8.48 (d, 1H), 8.08 (d, 1H), 8.00 (dd, 1H), 7.84 (ddd, 1H), 7.69 (ddd, 1H), 4.46 (d, 1H), 4.31 (s, 1H), 4.14 (s, 1H), 3.77-3.68 (m, 1H), 3.51-3.34 (m, 2H), 3.01 (q, 3H), 2.91 (s, 2H), 2.81 (s, 6H), 2.03 (ddd, 1H), 1.83-1.77 (m, 2H), 1.59-1.45 (m, 5H), 1.39-1.29 (m, 11H), 0.99 (d, 2H), 0.96 (s, 2H).

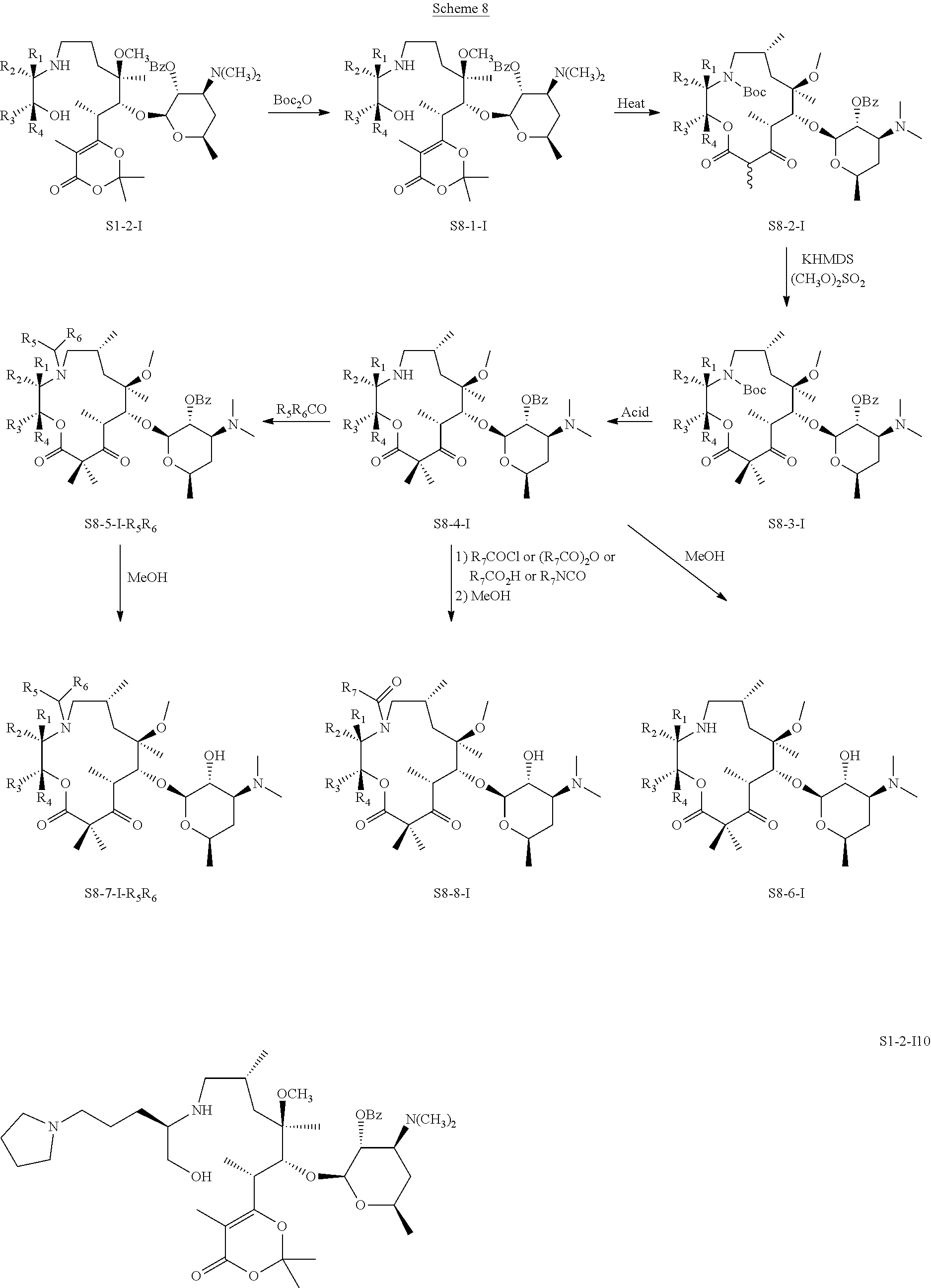

### (2S,3R,4S,6R)-4-(Dimethylamino)-2-(((2R,3R,4R,6R)-7-(((R)-1-hydroxy-5-(pyrrolidin-1-yl)pentan-2-yl)amino)-4-methoxy-4,6-dimethyl-2-(2,2,5-trimethyl-4-oxo-4H-1,3-dioxin-6-yl)heptan-3-yl)oxy)-6-methyltetrahydro-2H-pyran-3-yl benzoate (S1-2-I10)

S1-1 (0.675 g, 1.14 mmol) and (R)-2-amino-5-(pyrrolidin-1-yl)pentan-1-ol (I10, 234 mg, 1.36 mmol) were dissolved in EtOH (5 mL), and Ti(OEt)4 (0.72 mL, 2.96 mmol) was added. After 30 min, a small aliquot was removed from the reaction mixture and was added to a suspension of a small amount of NaBH₄ in MeOH. LC/MS analysis showed complete conversion. NaBH₄ (107 mg, 3.42 mmol) was added. When gas evolution ceased, 30% aqueous NH₄OH (3 mL) was added, and the mixture was filtered through a pad of Celite®, washing with EtOAc. The filtrate was washed with brine, was dried over Na₂SO₄, was filtered, and was concentrated to give S1-2-I10. The material was used without further purification. MS (ESI+) m/z: 746.49 [M+H]+, 374.00 [M+2H]2+249.66 [M+3H]<.

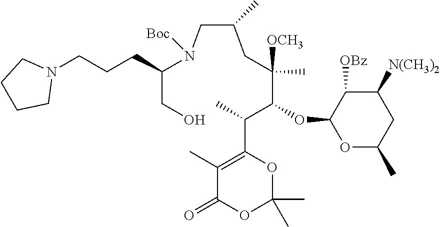

### (2S,3R,4S,6R)-2-(((2R,3R,4R,6R)-7-((tert-Butoxycarbonyl)((R)-1-hydroxy-5-(pyrrolidin-1-yl)pentan-2-yl)amino)-4-methoxy-4,6-dimethyl-2-(2,2,5-trimethyl-4-oxo-4H-1,3-dioxin-6-yl)heptan-3-yl)oxy)-4-(dimethylamino)-6-methyltetrahydro-2H-pyran-3-yl benzoate (S8-1-I10)

In a 40 mL vial was a solution of S1-2-I10 (850 mg, 1.13 mmol) in dichloromethane (5 mL) to give a yellow solution which was stirred at rt. Boc2O (0.33 mL, 1.46 mmol) was added In an portion and allowed to stir at rt for 2 hours. The reaction was diluted with dichloromethane and poured into satd aq NaHCO₃. The aqueous phase was extracted with dichloromethane and the combined organic phases were dried over MgSO4, filtered and concentrated. The residue was purified on 24 g silica gel (elution with 0-6% MeOH-dichloromethane) to give S8-1-I10 (520 mg, 54% in two steps). MS (ESI+) m/z: 846.5 [M+H]+, 423.8 [M+2H]2+.

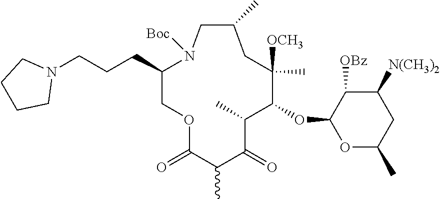

### tert-Butyl (3R,6R,8R,9R,10R)-9-(((2S,3R,4S,6R)-3-(benzoyloxy)-4-(dimethylamino)-6-methyltetrahydro-2H-pyran-2-yl)oxy)-8-methoxy-6,8,10,12-tetramethyl-11,13-dioxo-3-(3-(pyrrolidin-1-yl)propyl)-1-oxa-4-azacyclotridecane-4-carboxylate (S8-2-I10)

The flask was fitted with a reflux condenser and the condenser was flame dried under vacuum, allowed to cool and backfilled with nitrogen. The solution of S8-2-I10 (520 mg, 0.614 mmol) in chlorobenzene (150 mL) was added via cannula and the flask was placed under mild vacuum and sonicated for 2 minutes, then backfilled with nitrogen. The degassing procedure was repeated, then the mixture was heated at a bath temperature of 155 °C for 16 hours. The reaction was allowed to cool to rt and was concentrated. The residue was purified on 24 g of silica gel (elution with 0-10% MeOH-dichloromethane+0.5% of 30% aq NH₄OH) to give S8-2-I10 (395 mg, 82%). MS (ESI+) m/z: 394.8 [M+2H]²+, 788.5 [M+H]-.

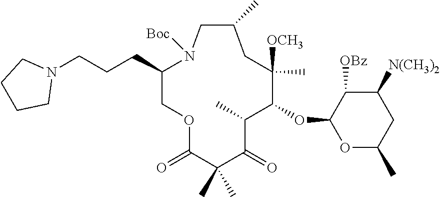

### tert-Butyl (3R,6R,8R,9R,10R)-9-(((2S,3R,4S,6R)-3-(benzoyloxy)-4-(dimethylamino)-6-methyltetrahydro-2H-pyran-2-yl)oxy)-8-methoxy-6,8,10,12,12-pentamethyl-11,13-dioxo-3-(3-(pyrrolidin-1-yl)propyl)-1-oxa-4-azacyclotridecane-4-carboxylate (S8-3-I10)

In a 20 mL vial was a solution of S8-2-I10 (360 mg, 0.46 mmol) in 1,2-dimethoxyethane (5 mL) precooled at −60 °C. KHMDS (0.68 mL, 0.68 mmol) was added dropwise. The reaction mixture was stirred at −60 °C for 20 min. Then Me₂SO₄ (65 μL, 0.68 mmol) was added. The reaction mixture was allowed to warm to −15 °C. LC/MS shows full conversion. The reaction was quenched by adding trimethylamine (0.88 mL) and the resulting mixture was diluted with dichloromethane and saturated NaHCO₃ was added. The aqueous layer was extracted with dichloromethane and the combined organic layers were dried over MgSO4, filtered and concentrated. The residue was purified on 4 g of silica gel (elution with 0-10% MeOH-dichloromethane+0.5% of 30% aq NH₄OH) to give S8-3-I10 (145 mg, 40%). MS (ESI+) m/z: 401.8 [M+2H]2+, 802.5 [M+H]+.

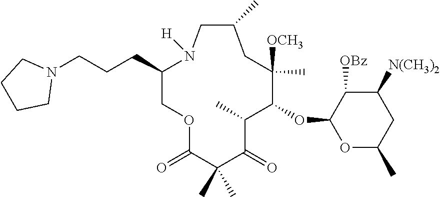

### (2S,3R,4S,6R)-4-(Dimethylamino)-2-(((3R,6R,8R,9R,10R)-8-methoxy-6,8,10,12,12-pentamethyl-11,13-dioxo-3-(3-(pyrrolidin-1-yl)propyl)-1-oxa-4-azacyclotridecan-9-yl)oxy)-6-methyltetrahydro-2H-pyran-3-yl benzoate (S8-4-I10)

In a 20 mL flask was a solution of S8-3-I10 (135 mg, 0.17 mmol) in 1.5 mL of DCM at rt. TFA (0.52 mL, 6.74 mmol) was added and the mixture was stirred at rt. The reaction was complete by UPLC. The mixture was diluted with 30 mL of DCM and 30 mL of said aq NaHCO₃ was added. The aqueous phase was extracted 3×w/DCM and the combined organic phases were dried over MgSO4, filtered and concentrated to give S8-4-I10. The product was used as is without further purification. MS (ESI+) m/z: 234.8 [M+3H]3+, 351.8 [M+2H]2+, 702.5 [M+H]+.

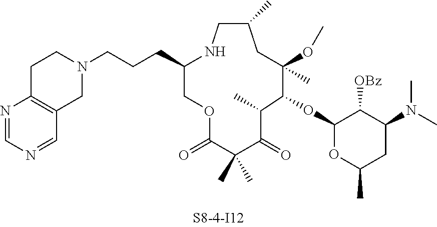

### (2S,3R,4S,6R)-2-(((3R,6R,8R,9R,10R)-3-(3-(7,8-dihydropyrido[4,3-d]pyrimidin-6(5H)-yl)propyl)-8-methoxy-6,8,10,12,12-pentamethyl-11,13-dioxo-1-oxa-4-azacyclotridecan-9-yl)oxy)-4-(dimethylamino)-6-methyltetrahydro-2H-pyran-3-yl benzoate (S8-4-I12)

Prepared according to the methods of S8-4-I10, substituting intermediate 112 to give S8-4-I12. MS (ESI+) m/z: 383.58 [M+2H]2+, 766.00 [M+H]+.

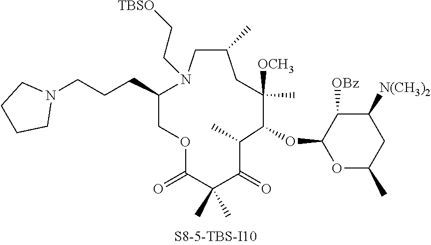

### (2S,3R,4S,6R)-2-(((3R,6R,8R,9R,10R)-4-(2-((tert-Butyldimethylsilyl)oxy)ethyl)-8-methoxy-6,8,10,12,12-pentamethyl-11,13-dioxo-3-(3-(pyrrolidin-1-yl)propyl)-1-oxa-4-azacyclotridecan-9-yl)oxy)-4-(dimethylamino)-6-methyltetrahydro-2H-pyran-3-yl benzoate (S8-5-TBS-I10)

Compound S8-4-I10 (105 mg, 0.15 mmol) was dissolved in dry methylene chloride (1 mL) and 2-((tert-butyldimethylsilyl)oxy)acetaldehyde (0.042 mL, 0.223 mmol) and AcOH (0.026 mL, 0.45 mmol) was added. Then NaBH(OAc)₃ (63 mg, 0.30 mmol) was added to the reaction mixture In an portion. The reaction was allowed to stir at rt for 2 h and LC/MS shows full conversion. The reaction was quenched by adding saturated NaHCO₃ (5 mL) and the aqueous layer was extracted with methylene chloride three times (10 mL). The combined organic layers were dried over MgSO4, filtered and concentrated. The residue was purified on 4 g of silica gel (elution with 0-10% MeOH-dichloromethane+0.5% of 30% aq NH₄OH) to give 61 mg of S8-5-TBS-I10 (48% yield). MS (ESI+) m/z: 287.5 [M+3H]3+, 430.8 [M+2H]2+, 860.6 [M+H]+.

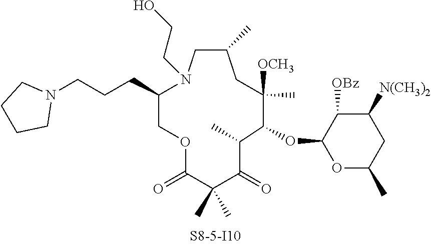

### (2S,3R,4S,6R)-4-(Dimethylamino)-2-(((3R,6R,8R,9R,10R)-4-(2-hydroxyethyl)-8-methoxy-6,8,10,12,12-pentamethyl-11,13-dioxo-3-(3-(pyrrolidin-1-yl)propyl)-1-oxa-4-azacyclotridecan-9-yl)oxy)-6-methyltetrahydro-2H-pyran-3-yl benzoate (S8-5-I10)

S8-5-TBS-I10 (61 mg, 0.071 mmol) was dissolved in dry THF (2 mL) and TBAF (IM in THF, 0.21 mL, 0.021 mmol) was added at room temperature. The reaction mixture was stirred at rt for 2 h and was concentrated. The residue was purified on 4 g of silica gel (elution with 0-20% MeOH— dichloromethane+0.5% of 30% aq NH₄OH) to give S8-5-I10 (46 mg, 87%). MS (ESI+) m/z: 249.5 [M+3H]3+, 373.8 [M+2H]2+, 746.5 [M+H]+.

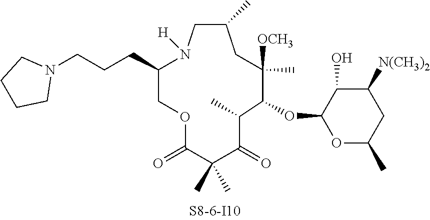

### (3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-3-(3-(pyrrolidin-1-yl)propyl)-1-oxa-4-azacyclotridecane-11,13-dione (S8-6-I10)

S8-4-I10 (25 mg, 0.036 mmol) was dissolved in MeOH (1 mL), and the reaction mixture was heated to 40 °C (external temp.) overnight. The reaction mixture was cooled to rt and was concentrated under reduced pressure. The material was purified by HPLC (Atlantis T3 column, 5-30% MeCN-water-0.1% HCO2H) to give 6.52 mg of S8-6-I10 as a formate salt. MS (ESI+) m/z: 200.1 [M+3H]3+, 299.7 [M+2H]2+, 598.4 [M+H]+; 1H NMR (400 MHz, Methanol-d) δ 8.56 (s, 1H), 4.39 (d, 1H), 4.13 (d, 1H), 3.87 (dd, 1H), 3.63 (ddt, 1H), 3.49 (dt, 1H), 3.35-3.27 (m, 3H), 3.11 (d, 1H), 3.10-2.96 (m, 3H), 2.94 (d, 5H), 2.86 (s, 1H), 2.69 (dd, 1H), 2.50 (s, 4H), 2.44 (t, 3H), 2.04-1.96 (m, 4H), 1.91-1.78 (m, 4H), 1.71-1.57 (m, 3H), 1.48 (s, 3H), 1.44 (d, 1H), 1.41-1.32 (m, 8H), 1.27 (d, 5H), 1.02 (d, 3H).

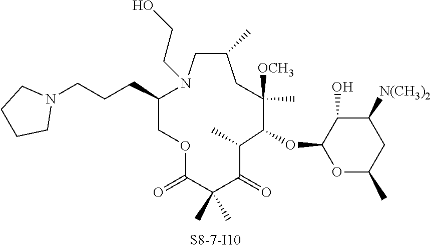

### (3R,6R,8R,9R,10R)-9-(((2S,3R,4S,6R)-4-(Dimethylamino)-3-hydroxy-6-methyltetrahydro-2H-pyran-2-yl)oxy)-4-(2-hydroxyethyl)-8-methoxy-6,8,10,12,12-pentamethyl-3-(3-(pyrrolidin-1-yl)propyl)-1-oxa-4-azacyclotridecane-11,13-dione (S7-7-I10)

Prepared according to the method of S8-6-110 and starting from S8-5-110 to provide the title compound as a formate salt. MS (ESI+) m/z: 214.8 [M+3H]3+, 321.8 [M+2H]2+, 642.5 [M+H]+; 1H NMR (400 MHz, Methanol-d) δ 8.49 (s, 2H), 4.52 (s, 1H), 4.44 (d, 1H), 4.04 (d, 1H), 3.84 (s, 1H), 3.70 (ddd, 1H), 3.55 (d, 3H), 3.49-3.33 (m, 2H), 3.33 (s, 3H), 3.30 (p, 1H), 3.22-3.08 (m, 2H), 3.03 (s, 1H), 2.81 (s, 6H), 2.55 (s, 2H), 2.26 (s, 1H), 2.08 (d, 1H), 2.09-2.00 (m, 3H), 2.03-1.91 (m, 1H), 1.83-1.74 (m, 2H), 1.58-1.45 (m, 1H), 1.45 (s, 2H), 1.39 (s, 1H), 1.35 (s, 2H), 1.33-1.24 (m, 8H), 1.15 (s, 1H), 0.84 (d, 3H).

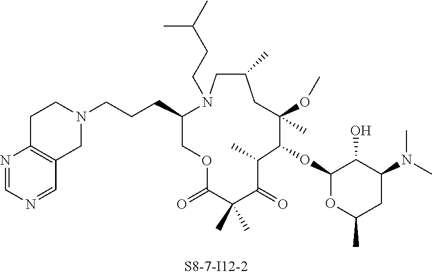

### (3R,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-4-isopentyl-8-methoxy-6,8,10,12,12-pentamethyl-1-oxa-4-azacyclotridecane-11,13-dione (S8-7-I12-2)

Prepared according to the methods of S8-7-I10, starting from S8-4-I12, and substituting 3-methylbutanal to provide 16.47 mg of S8-7-I12-2 as a formate salt. MS (ESI+) m/z: 366.51 [M+2H]2+, 731.94 [M+H]+; ¹H NMR (400 MHz, Methanol-d₄) δ 8.94 (s, 1H), 8.55 (s, 1H), 8.51 (s, 2H), 4.47 (d, 1H), 4.22 (s, 1H), 3.74 (q, 4H), 3.51 (dd, 1H), 3.41 (ddd, 2H), 3.04 (t, 3H), 3.01-2.89 (m, 6H), 2.84 (s, 7H), 2.78-2.61 (m, 3H), 2.05 (d, 3H), 1.87-1.62 (m, 5H), 1.60-1.54 (m, 2H), 1.52 (s, 6H), 1.38 (d, 5H), 1.34 (t, 8H), 0.94 (t, 9H).

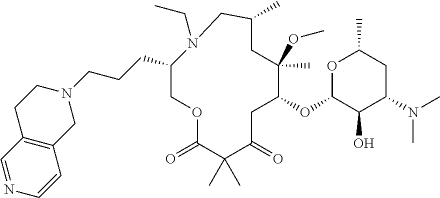 [SMILES: CCN1C[C@H](C)C[C@@](C)(OC)[C@H](O[C@@H]2O[C@H](C)C[C@H](N(C)C)[C@H]2O)CC(=O)C(C)(C)C(=O)OC[C@@H]1CCCN1CCc2cnccc2C1]

### (3S,6R,8R,9R,10R)-3-(3-(3,4-dihydro-2,6-naphthyridin-2(1H)-yl)propyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione

Prepared according to the methods of S6-3-I1-1 from S2-1-I1-2 and 1,2,3,4-tetrahydro-2,6-naphthyridine to provide the title compound as a formate salt. MS (ESI+) m/z: 689.3 [M+H]+; ¹H NMR (400 MHz, Methanol-d4) δ 8.40 (s, 2.6H), 8.33 (s, 1H), 8.26 (d, 1H), 7.17 (d, 1H), 4.66-4.57 (m, 1H), 4.52-4.40 (m, 2H), 4.27 (br d, 1H), 4.03 (br d, 1H), 3.81-3.67 (m, 3H), 3.63-3.50 (m, 1H), 3.49-3.34 (m, 3H), 3.25-3.07 (m, 2H), 3.07-2.94 (m, 5H), 2.92-2.77 (m, 9H), 2.69 (br t, 2H), 2.17 (br d, 1H), 2.08-2.01 (m, 1H), 1.95 (br d, 1H), 1.88-1.70 (m, 3H), 1.63 (br d, 1H), 1.58-1.50 (m, 1H), 1.49-1.44 (m, 3H), 1.44-1.27 (m, 16H), 1.06 (br d, 3H).

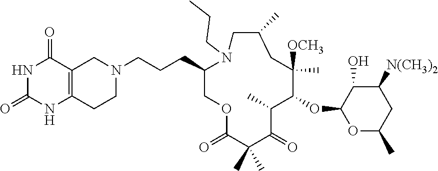

### (3R,6R,8R,9R,10R)-9-(((2S,3R,4S,6R)-4-(dimethylamino)-3-hydroxy-6-methyltetrahydro-2H-pyran-2-yl)oxy)-3-(3-(2,4-dioxo-1,3,4,5,7,8-hexahydropyrido[4,3-d]pyrimidin-6(2H)-yl)propyl)-8-methoxy-6,8,10,12,12-pentamethyl-4-propyl-1-oxa-4-azacyclotridecane-11,13-dione

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and hexahydropyrido[4,3-d]pyrimidine-2,4(1H,3H)-dione.

### Biological Examples

We synthesized a series of macrolide compounds with the aim of targeting the mammalian ribosome to modulate the read-through of premature stop codons. In the present study, we explored the potential of 13-membered macrolides to induce read-through of premature stop codons in the APC gene.

Human colorectal carcinoma SW403 and SW1417 cells harboring PTC mutations in the APC gene treated with a 13-membered macrolide as disclosed herein had reduced levels of nuclear b-catenin and c-myc, indicating that the macrolide mediated read-through of premature stop codons to produce bioactive APC protein inhibiting the b-catenin/wnt-pathway. In a mouse model of adenomatous polyposis coli, treatment of APC^{min} mice with a macrolide as disclosed herein caused a in significant decrease in intestinal polyps, adenomas and associated anemia, resulting in increased survival. Immuno-histochemistry revealed decreased nuclear b-catenin staining in the epithelial cells of the polyps in treated APC^{min} mice confirming the impact on b-catenin/wnt-pathway. These results indicate that macrolides as disclosed herein may have therapeutic potential for the treatment of FAP caused by nonsense mutations in the APC gene.

### Materials and Methods

Cell lines. Human colon cancer cell SW1417 and SW403 were purchased from American Type Culture Collection (ATCC, Rockville, MD, USA). Cells were cultured in Dulbecco Modified Eagle Medium (DMEM High Glucose with GlutaMAX, Gibco, Thermo Fisher Scientific, Waltham, MA) supplemented with 10% Fetal Bovine serum (FBS, heat inactivated, Gibco) and 1% Penicillin-Streptomycin (Gibco) and maintained at 37 °C in a humidified incubator supplied with 5% of CO₂. Cells were passaged weekly with trypsin.

### Cell Growth Inhibition

Western blots. Cells were plated at a density of 4×10⁶ cells/100 mm dish. After 48 hours, cells were treated with either DMSO or a compound of the invention as recited in Table A (50 mM). 48 hours post treatment, cell pellets were harvested and split into two aliquots. One aliquot was used to prepare whole cell lysates (RIPA lysis and Extraction Buffer, Thermo Fisher Scientific), the other aliquot was used to generate nuclear and cytoplasmic protein extracts (NE-PER Nuclear and Cytoplasmic Extraction Reagents, Thermo Fisher Scientific). Proteins were separated on 4-12% gradient gels (NuPAGE Bis-Tris protein gels, Invitrogen, Thermo Fisher Scientific) using SDS running buffer (NuPAGE MES SDS Running Buffer, Invitrogen). Primary antibodies used include: non-phosphorylated b-catenin (Ser33/37/Thr41) (1:1000, catalogue 8814, Cell Signaling Technology, Danvers, MA): c-myc (1:1000, catalogue ab32072, Abcam, Waltham, MA), MEK1/2 (1:1000, catalogue 8727, Cell Signaling Technology), Histone H3 (1:1000, catalogue 4499, Cell Signaling Technology) and a-tubulin (1:10,000, catalogue T6199, MilliporeSigma, Burlington, MA).

Animal studies. Pharmacokinetic studies were performed at WuXi AppTec Laboratory Testing Division (Cranbury, NJ) in male CD-1 mice sourced from Hilltop Lab Animals, Inc. (Scottdale, PA) 6-8 weeks of age. Mice were allowed free access to water and rodent chow pellets (LabDiet, Certified Rodent Diet #5002). Animals received a single oral dose of a compound of the invention as recited in Table A (100 mg/kg body weight) from a sterile stock solution of 10 mg/ml saline. Blood samples were collected in EDTA tubes from peripheral veins at 0.083, 0.250, 0.500, 1.00, 2.00, 4.00, 8.00 and 24.0 hour time points post-dosing and processed for plasma. After the last blood collection, mice were euthanized, backs were shaved and a 1 cm² skin sample was excised and weighed. A 2 cm section of jejunum was harvested, rinsed with saline and weighed. Tissue samples and plasma was stored at −70 °C. Thawed skin samples were finely minced, extracted with acetonitrile, centrifuged at 3000×g and supernatants were collected for LC-MS/MS analysis. Water was added to thawed intestinal tissue at a 6:1 ratio (ml water/mg tissue), homogenized and processed for LC-MS/MS analysis. A SCTEX Triple Quad 6500+LC-MS/MS system (SCIEX, Framingham, MA) was used to analyze plasma, jejunum homogenate and skin/acetonitrile samples. Briefly, proteins were precipitated using IS solution (100 ng/mL Labetalol & 100 ng/mL Tolbutamide in Acetonitrile) and centrifuged at 3900×g. Supernatant was mixed with an equal volume of water and 2.00-4.00 mL was injected for LC-MS/MS analysis. Calibration curves in the range of 1.00-3000 ng/ml were generated in corresponding tissue preparations from naïve CD-1 mice. Data analysis was performed using Phoenix WinNonlin software (Certara, Princeton, NJ).

APC^{min} mice of C57BL/6 strain were obtained from Jackson Laboratory (Bar Harbor, ME) and female APC^{min} mice 10 weeks of age with body weight greater than 15 g were used. Mice were housed using individually ventilated caging system and allowed free access to sterile rodent chow (Teklad 2919) and water. Clinical observations and body weights were recorded daily and mice with body weight loss greater than 20% were removed from study. Study was conducted under compliance of the UK Animals Scientific Procedures Act 1986 and was performed at Crown Biosciences UK Ltd (Loughborough, UK). The compound was formulated in sterile saline to a working concentration of 5 mg/ml. Mice were treated daily with oral doses of the compound (50, 25 or 12.5 mg/kg body weight) or saline for 8 weeks. Drug was well tolerated with no adverse events noted. As animals became moribund or at the end of study, mice were euthanized; a full necropsy was performed; spleens weighed; and the complete gastro-intestinal tract was removed for analysis. Whole blood was collected in EDTA tubes and complete blood counts (CBC) were as performed on a Woodley InSight 5 Diff Retic Haematology Analyzer and compared to normal murine CBC ranges (Pinmoore Animal Laboratory Services Limited, Cheshire, UK).

Polyp scoring, histo-pathology and immuno-histochemistry. The excised intestinal tract (small and large bowel) was flushed with PBS and sliced longitudinally; 10 cm sections were visually inspected; and polyps were enumerated. The mid-sections of the small intestines were fixed in 10% formalin; fashioned into swiss rolls; processed into FFPE blocks; and 4 mm sections were used for analysis. A Tissue-Tek Prisma Plus autostainer (Sakura Finetek USA, Torrance, CA) was used for hematoxylin and eosin (H&E) staining. Sections were scanned with Pannoramic Digital Slide Scanners at 40× magnification (3DHISTECH, Pannoramic SCAN, Budapest, Hungary). Analysis and quantification of H&E stained slides were performed by HALO Image Analysis software (Indica Labs, Albuquerque, NM, USA); lesions were identified by nucleus to cytoplasmic staining ratio; and lesion area was calculated (Percent lesion area=lesion area/small intestine area X 100) [31, 32]. Identification of adenoma regions was performed by a board certified pathologist. Immuno-histochemistry (IHC) was performed on sections from FFPE blocks using the Leica BOND-RXm Automated Stainer (Deer Park, IL, USA), set at the following antigen retrieval conditions: citrate, pH 6.0; 100 °C; 20 min; primary antibodies 1:100 dilution. Rabbit IgG mAb for b-catenin was obtained from Cell Signaling Technology. A polyclonal anti-rabbit IgG conjugated to horseradish peroxidase was used for detection (Leica Biosystems, Buffalo Grove, IL). Non-specific rabbit IgG (Abcam) was used for an isotype control. Sections were rinsed and counterstained with H&E.

Statistical analysis. GraphPad Prism software version 9 (GraphPad Software, La Jolla, CA) was used for data analysis and the generation of graphs. Mean values are plotted with the standard deviation (SD) and standard error of the mean (SEM) indicated by bars. The unpaired student t-test was used to compare the means of the treatment groups to control group. In survival analysis, the log-rank test was used to determine significance between the control and treatment groups. p-values less than 0.05 were considered significant.

### Results

### Compound Growth Inhibition of Human Colon Carcinoma Cells.

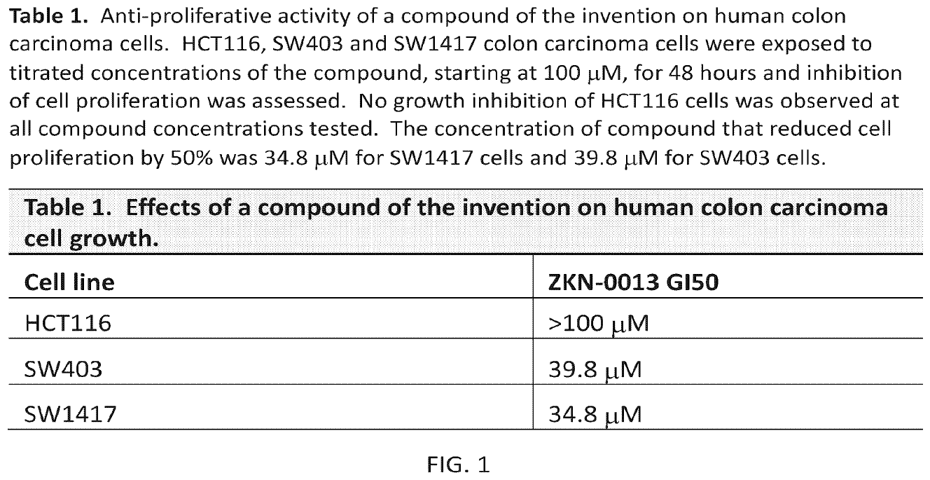

Ribosomal targeting antibiotics that promote read-through of premature stop codons can inhibit the proliferation of tumor cells harboring nonsense mutations in the APC gene through the down-regulation of b-catenin/c-myc signaling [23, 24, 33]. SW1417 and SW403 human colon carcinoma cells contain a nonsense mutation in the APC gene (R1450* and S1278*, respectively) resulting in a non-functional, truncated APC protein and HCT116 human colon carcinoma cells contain the wild-type APC gene [17]. A growth inhibition assay was conducted to evaluate the anti-proliferative activity of a compound as disclosed herein on these 3 tumor cell lines. Tumor cells were cultured in the presence of titrated concentrations of a compound as disclosed herein and the drug concentration that inhibited 50% of cell growth as compared to vehicle treated cells (G150) is shown in Table 1 (FIG. 1). The compound had no effect on the growth of HCT116 cells at all the concentrations tested (up to 100 mM) and the GI50s for SW1417 and SW403 cells were 34.8 and 39.8 mM, respectively.

Treatment of Human Colon Cancer Cells with a Compound as Disclosed Herein Results in Decreased Levels of Nuclear b-Catenin and c-Myc.

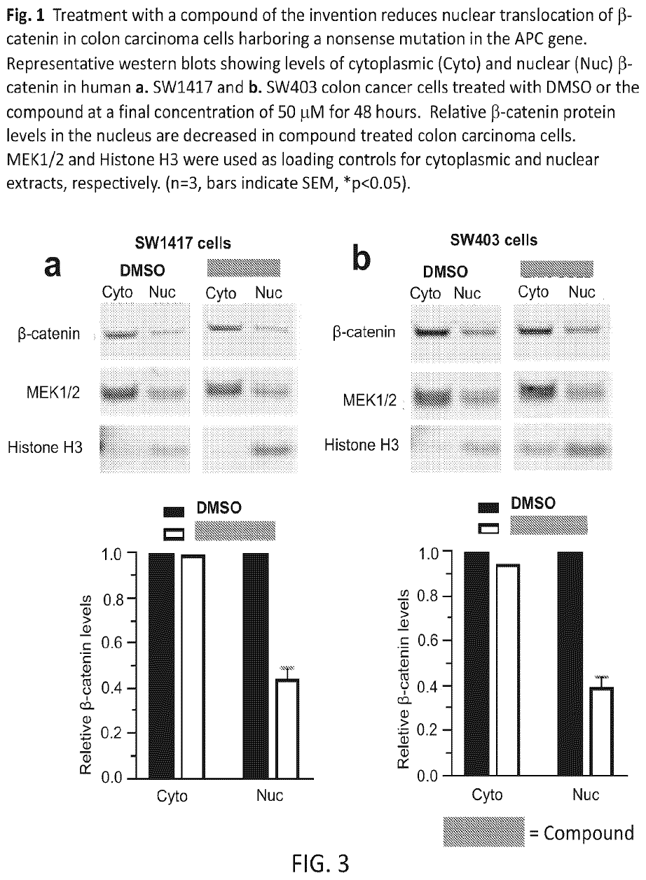

Adenomatous polyposis coli is a large, complex protein (312 kDa) which is directly involved in ubiquitin degradation of phosphorylated b-catenin in the cytoplasm [34]. In the absence of APC activity, there is increased translocation of b-catenin to the nucleus. Therefore, the amount of nuclear b-catenin relative to that in the cytoplasm is used as an indicator of the level of functional APC, particularly since there is a lack of reliable, commercially available antibodies for the detection of APC [35, 36]. SW403 and SW1417 cell were treated with 50 mM of the compound for 48 hours and b-catenin levels were determined by western blot in the nuclear and cytoplasmic extracts. As shown in FIG. 1 (FIG. 3), colon carcinoma cells exhibit a significant reduction of nuclear b-catenin accumulation in response to compound exposure. The nuclear level of b-catenin was reduced by 40% in SW1417 and SW403 treated cells as compared to DMSO treated cells. This indicates increased levels of functional APC resulting from the compound mediated read-through of APC nonsense mutations. Cytoplasmic levels of b-catenin in cells treated with DMSO or the compound was similar.

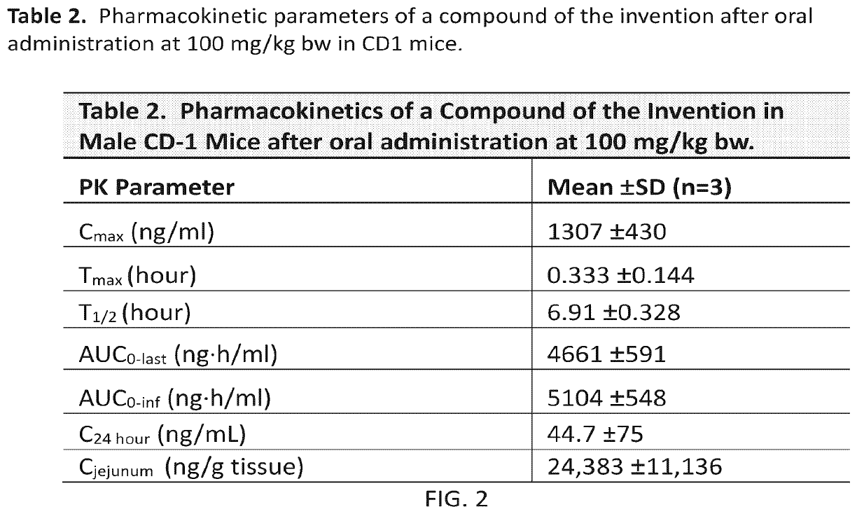

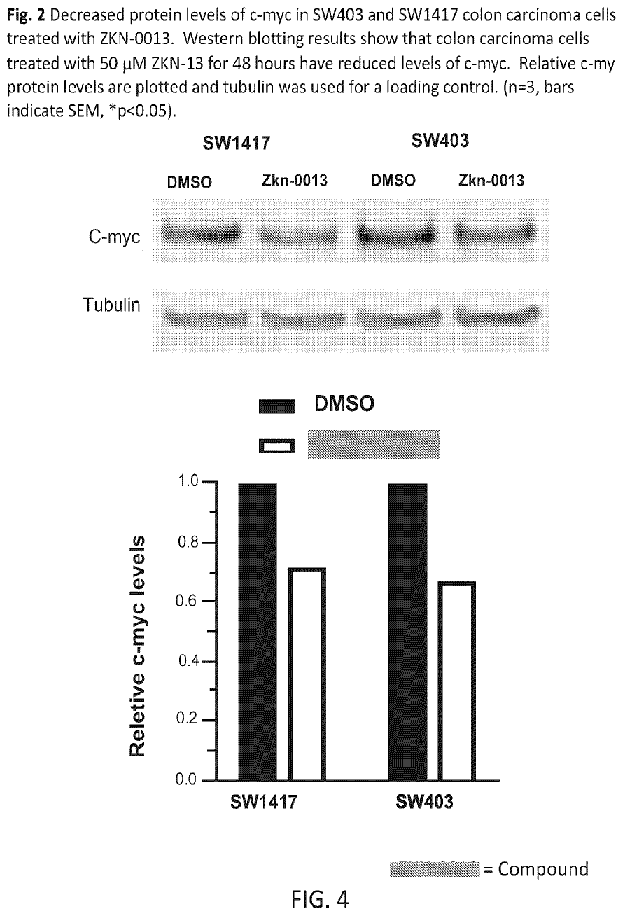

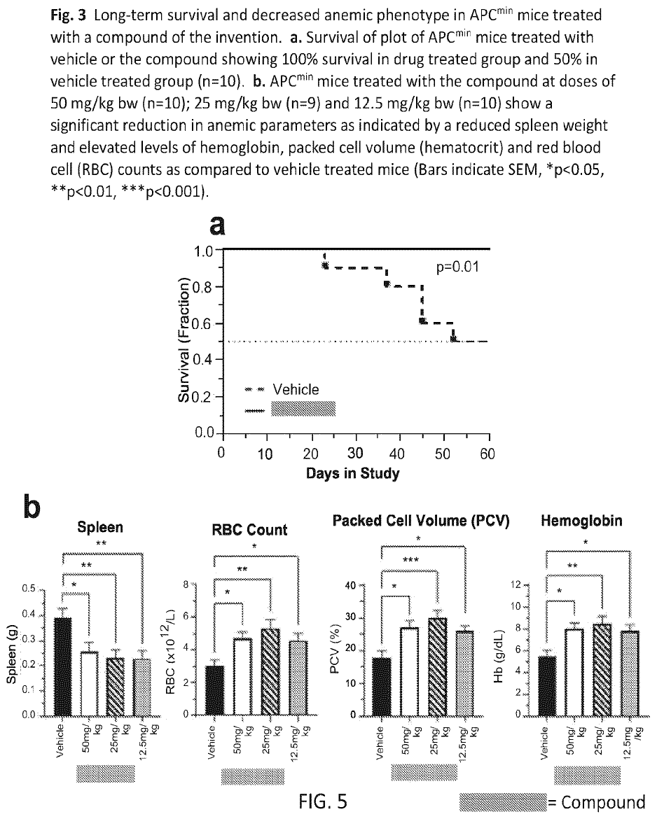

Expression of the c-myc gene is transcriptionally co-regulated by b-catenin [8, 37]. As shown in FIG. 2 (FIG. 4), the compound of the invention as recited in Table A (50 mM, 48 hours) treatment reduced c-myc protein levels by 25% in SW1417 cells and by 30% in SW403 cells relative to cells treated with DMSO. Thus, providing additional evidence that compound treatment leads to increased levels of active APC, promoting cytoplasmic degradation of b-catenin and a reduction of nuclear levels of b-catenin, resulting in decreased expression of c-myc.

Treatment with a Compound as Disclosed Herein Improves the Survival of APC^{min} Mice and Reduces Anemic Phenotype.

A single dose mouse study was conducted to determine the pharmacokinetics and exposure of a compound as disclosed herein in intestinal tissue after oral administration. Male CD-1 mice (n=3) received an oral dose of the compound at 100 mg/kg body weight and drug plasma levels were measured at several time points in a 24 hour period. The results are summarized in Table 2 (FIG. 2). Mean plasma C-max was 1307 ng/ml (+430 SD) which peaked approximately 15 minutes after dosing. At the end of the 24 hour study period, the mean plasma level of the compound was 44.7 ng/ml and the mean drug concentration in the jejunum was 24,383 ng/g of tissue. This data demonstrates that the compound can achieve intestinal exposure of approximately 35 mM after single, oral dose where it can affect disease progression. The observed plasma half-life; effective tissue penetration; and high tissue-to-plasma concentration ratios are consistent with pharmacokinetics of the macrolide class of drugs [38].

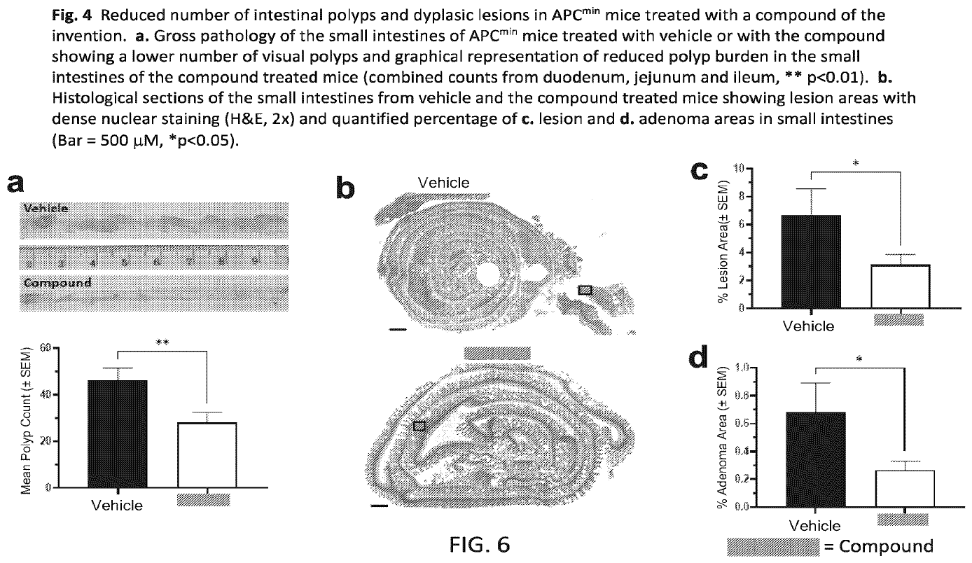

A well-established model of FAP is the APC^{min} mouse in which the human APC homologue gene (min) contains a nonsense mutation generating a non-functional, truncated protein. APC^{min} mice develop numerous small intestinal adenomas as the primary phenotype and anemia as a secondary, lethal condition resulting in an average lifespan of 120 days [18-20]. The majority of adenomas are present in APC^{min} mice at 5-8 weeks of age and the number of adenomas does not significantly increase thereafter [39]. In an 8-week efficacy study, APC^{min} mice were treated daily with oral doses of a compound as disclosed herein (50 mg/kg body weight) or vehicle saline. During the third week of the study, mice in the vehicle control group began to show severe clinical signs of disease progression and had to be terminated. Only 5 vehicle treated mice (50%) remained in the study at the endpoint. In contrast, all the mice treated with the compound remained on study for the full duration. Similar efficacy results were obtained in a repeat study using daily oral doses of the compound at 50, 25 and 12.5 mg/kg body weight with 100% survival achieved at the lowest dose used in the 8-week study. Anemia resulting from chronic blood loss from the numerous intestinal lesions in APC^{min} mice is considered to be the major cause of animal death [18]. Compound treatment significantly improved the anemia associated with the APC^{min} model as indicated by an approximately 35% reduction of splenomegaly, and increased levels of hemoglobin (approximately 45%), packed cell volume (approximately 55%) and red blood cell counts (approximately 60%) in the mice at all 3 dose levels tested (FIG. 3B)(FIG. 5bB. Clinical observations, consistent with the APC^{min} model include pale limb extremities and discolored feces, were reported in both groups and body weight gain was comparable in vehicle and the treatment groups through-out the course of the study.

Compound treatment reduces the number of dysplastic intestinal polyps and lesion area in APC^{min} mice.

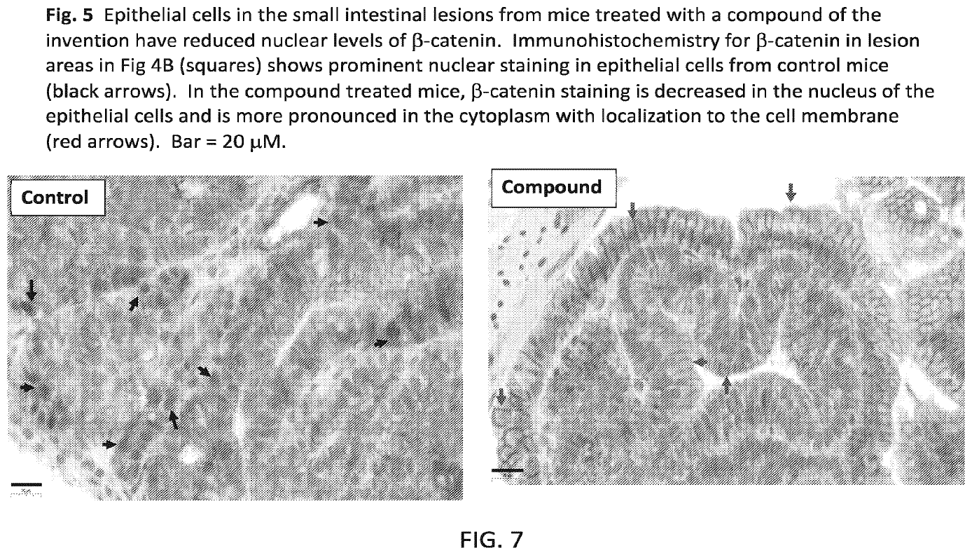

As mice became moribund or at the end of the study in which mice were treated with 50 mg/kg bw a compound as disclosed herein, the entire gastric-intestinal track was removed and polyps were manually counted. The total number of visual polyps in the small intestines of mice treated with the compound was significantly less than that of control mice with a 39% reduction in polyp number (FIG. 4A)(FIG. 6A). There were very few polyps detected in the large intestines of the mice from either group, which is characteristic of the model (data not shown). Hematoxylin and eosin stained sections from the small intestines of control and the compound treated mice are shown in FIG. 4B and was used for histo-pathological analysis. Total lesion area (polyp, adenoma and carcinoma) was decreased by 52% and the area of adenomas was also reduced by 60% in compound treated mice (FIG. 4B)(FIG. 6B). Immuno-histochemistry, shown in FIG. 5 (FIG. 7), revealed prominent nuclear b-catenin staining in numerous epithelial cells in the lesions of saline treated mice. There were noticeably fewer epithelial cells with nuclear b-catenin staining in the lesions from compound treated mice; numerous epithelial cells in these lesions presented with membrane-associated b-catenin staining; and retained their columnar morphology (FIG. 5)(FIG. 7).

### Discussion.

Recent studies have demonstrated that read-through of nonsense mutations can be induced by some macrolide antibiotics and aminoglycosides in human cancer cells and partially restore the synthesis of functional, full-length proteins such as p53 and APC [23, 24, 40, 41]. As a tumor suppressor, a major role of APC is the formation of a core complex with Axin, Ser/Thr kinases glycogen synthase kinase 3 and casein kinase I for the ubiquitination and proteasomal degradation of cytoplasmic b-catenin [34]. Consequently, there is a decrease of nuclear translocation of b-catenin and reduced transcription of b-catenin/wnt pathway genes including oncogenic c-myc [16, 34]. The APC protein consists of multiple functional domains. The regions between amino acids 1020-1169 and 1342-2075 are essential for b-catenin binding and degradation [8-10]. Nonsense mutations often result in the synthesis of a truncated APC protein lacking these domains which are unable to mediate b-catenin degradation.

Aminoglycoside and macrolide induced read-through typically results in restoration of 2-10% of normal protein levels. It has been reported that this amount of full-length protein is sufficient to restore function of APC [40]. However, detection of small amounts of a large protein such as APC by traditional antibody based methods is challenging. Recently, the sensitivity and specificity of commercially available anti-APC antibodies has come into question; therefore, we quantified the relative levels of cytoplasmic and nuclear b-catenin as a measure of functional activity of APC protein [36]. Our results from western blot analysis and immuno-histochemistry, provide evidence that treatment with a compound of the invention as recited in Table A of human colon carcinoma cells and adenomatous epithelial cells in APC^{min} mice, both of which contain a nonsense mutation in the APC gene, causes a reduction of nuclear b-catenin. Furthermore, in the colon carcinoma cells, this decrease of nuclear b-catenin results in a subsequent reduction of c-myc protein levels and inhibition of cell proliferation. This is consistent with other studies in which macrolide antibiotic and aminoglycoside treatment of cultured colorectal cancer cells containing APC nonsense mutations resulted in decreases in cell growth and nuclear levels of b-catenin [23, 24, 33]. In these studies, tylosin treatment of SW1417 colon carcinoma cells promoted a degree of read-through of the nonsense mutation leading to the synthesis of a low level of full-length, active APC protein [23]. In studies aimed at assessing aminoglycoside mediated read-through of several APC nonsense mutations Floquet et al. utilized a luciferase based reporter vector to measure the interaction between active APC and b-catenin and showed that the L360X stop codon was most susceptible to gentamicin mediated read-through [40]. It was noted that this indirect approach alleviated the potential difficulties in detecting low levels of APC protein which may be sufficient for the biological activity but not detectable by western blotting [40]. It is possible that restoration of even 1% of normal protein function may be adequate to lessen the severity of diseases associated with APC dysfunction.

In certain disease animal models that are associated with inactive APC, such as the APC^{min} mouse or human colon carcinoma tumor xenografts, suppression of premature translation termination of APC mRNA has shown to ameliorate disease in terms of reduced intestinal polyps and tumor growth inhibition [23, 24]. The results of our in vivo studies using the APC^{min} model are similar in that we show treatment with the novel macrolide compound of the invention as recited in Table A, reduced the number of intestinal polyps and the neoplastic appearance of intestinal lesions. Moreover, 100% of the APC^{min} mice treated with a compound of the invention as recited in Table A survived the 8-week study with no overt clinical signs of disease. In the control group, adverse clinical observations were noted by the third week of the study and only 50% of the vehicle treated mice remained at the end of the study. Additionally, compound treatment reduced the number of visual polyps in the small intestines by 39% which is a major contributing factor to anemia and the survival of the treated APC^{min} mice [20]. In a subsequent dose response study, efficacy was observed at all doses of the compound tested in APC^{min} mice. Even at the lowest daily dose of 12.5 mg/kg bw, anemia was alleviated as demonstrated by a 43% increase in hemoglobin levels and a 41% reduction of splenomegaly, contributing to long-term survival. It is important to note that our in vivo studies utilized older APC^{min} mice at an age where intestinal adenomas are pre-existing; therefore, providing pertinent support for the therapeutic use of a compound of the invention as recited in Table A for the treatment of FAP. In addition to its function in the nucleus, b-catenin is also an essential part of the adherens junction at the membrane where it binds to E-cadherin (PMID: 26240067). Dysregulation of Wnt pathway leading to increased nuclear localization of b-catenin also results in reduced cell-cell adhesion and cell loss from the villus (PMID: 26240067). As shown in FIG. 5, numerous epithelial cells in the adenomas of compound treated mice possess traditional columnar morphology and present with b-catenin staining aligned with the cell membrane. We have also observed elevated levels of e-cadherin associated with epithelial cell membranes of the adenomas of compound treated mice (data not shown). Collectively, compound treatment may enhance differentiation and inhibit or reverse disease progression as suggested by increased levels of membrane-associated b-catenin and e-cadherin where they form adherens junctions [42].

In eukaryotic cells, macrolide antibiotics target the proximal region of the nascent peptide exit tunnel on the large ribosomal subunit, interfering the peptidyl transferase center and slowing-down synthesis of the nascent polypeptide. This stalling allows for the insertion of a near-cognate tRNA amino acid at the premature termination codon site and continued translation [43, 44]. The traditional macrolide antibiotics that have demonstrated read-through activity are not suitable for chronic dosing that would be required to treat FAP patients. Prolonged use of these traditional macrolide antibiotics has the potential to result in cardiac and liver toxicity. These toxicities have been linked to the macrolide mediated inhibition of the hERG potassium channel in cardiomyocytes and the bile salt export pump (BSEP) in hepatocytes [28-30, 45-47]. We have developed and optimized novel macrolides as disclosed herein, with increased specificity for the mammalian ribosome and minimal off-target inhibition of hERG or BSEP activity (data not shown). Moreover, our pharmacokinetic data demonstrates that the compounds disclosed herein can penetrate and effectively accumulate in intestinal tissue after a single oral dose. This pharmacological profile with improved potency, minimal off-target activity and adequate target tissue exposure supports the application of the compounds disclosed herein for the chronic treatment of FAP patients.

The results of our studies demonstrate that macrolides as disclosed herein can suppress premature termination of protein translation induced by nonsense mutations in the APC gene, resulting in the restoration of active APC protein. In vitro, treatment with a compound as disclosed herein restored APC function in human colon carcinoma cells with known nonsense mutations in the APC gene. This was demonstrated by decreased levels of b-catenin in the nucleus of SW403 and SW1417 colon carcinoma cells treated with a compound as disclosed herein, indicative of APC mediated degradation of cytosolic b-catenin. Additionally, protein levels of c-myc, a downstream target of the b-catenin/wnt-pathway, was decreased in SW1417 and SW403 cell treated with as disclosed herein. These results provide strong evidence that compounds as disclosed herein promotes read-through of nonsense codons, restoring functional APC protein. In vivo efficacy of a compound as disclosed herein was demonstrated in the APC^{min} model using animals of age where polyps are already present. Treatment with a compound as disclosed herein significantly reduced the number of intestinal polyps and adenomatous tissue in these mice; alleviated the anemia associated with the model; leading to improved survival. Moreover, immuno-pathology showed numerous epithelial cells in the lesions from the mice treated with a compound as disclosed herein had reduced levels of nuclear b-catenin; an increase of cell membrane-associated b-catenin and possessed a more differentiated morphology. Taken together, our findings provide pre-clinical support to evaluate the therapeutic value of long-term use of compounds disclosed herein for the treatment of FAP patients.

### REFERENCES (47)

1 Half E, Bercovich D, Rozen P. Familial adenomatous polyposis. Orphanet J Rare Dis 2009; 4: 22.
2 Leoz M L, Carballal S, Moreira L, Ocana T, Balaguer F. The genetic basis of familial adenomatous polyposis and its implications for clinical practice and risk management. Appl Clin Genet 2015; 8: 95-107.
3 Fearnhead N S, Britton M P, Bodmer W F. The ABC of APC. Hum Mol Genet 2001; 10: 721-733.
4 De Queiroz Rossanese L B, De Lima Marson F A, Ribeiro J D, Coy C S, Bertuzzo C S. APC germline mutations in families with familial adenomatous polyposis. Oncol Rep 2013; 30: 2081-2088.
Polakis P. Mutations in the APC gene and their implications for protein structure and function. Curr Opin Genet Dev 1995; 5: 66-71.
6 Ripa R, Bisgaard M L, Bulow S, Nielsen F C. De novo mutations in familial adenomatous polyposis (FAP). Eur J Hum Genet 2002; 10: 631-637.
7 Galiatsatos P, Foulkes W D. Familial adenomatous polyposis. Am Gastroenterol 2006; 101: 385-398.
8 Hankey W, Frankel W L, Groden J. Functions of the APC tumor suppressor protein dependent and independent of canonical WNT signaling: implications for therapeutic targeting. Cancer Metastasis Rev 2018; 37: 159-172.
9 Senda T, Shimomura A, Iizuka-Kogo A. Adenomatous polyposis coli (Apc) tumor suppressor gene as a multifunctional gene. Anat Sci Int 2005; 80: 121-131.
10 Khalilzadeh S, Pakdaman S F, Momeni-Moghaddam M. The role of APC in Wnt/β-Catenin Pathway In gastric Cancer. The Cancer Press 2016; 2.
11 He T C, Sparks A B, Rago C, Hermeking H, Zawel L, da Costa L T et al. Identification of c-MYC as a target of the APC pathway. Science 1998; 281: 1509-1512.
12 Beroud C, Soussi T. APC gene: database of germline and somatic mutations in human tumors and cell lines. Nucleic Acids Res 1996; 24: 121-124.
13 Segditsas S, Tomlinson I. Colorectal cancer and genetic alterations in the Wnt pathway. Oncogene 2006; 25: 7531-7537.
14 Bian J, Dannappel M, Wan C, Firestein R. Transcriptional Regulation of Wnt/beta-Catenin Pathway in Colorectal Cancer. Cells 2020; 9.
15 Pai S G, Carneiro B A, Mota J M, Costa R, Leite C A, Barroso-Sousa R et al. Wnt/beta-catenin pathway: modulating anticancer immune response. J Hematol Oncol 2017; 10: 101.
16 Kolligs F T, Bommer G, Goke B. Wnt/beta-catenin/tcf signaling: a critical pathway in gastrointestinal tumorigenesis. Digestion 2002; 66: 131-144.
17 Rowan A J, Lamlum H, Ilyas M, Wheeler J, Straub J, Papadopoulou A et al. APC mutations in sporadic colorectal tumors: A mutational “hotspot” and interdependence of the “two hits”. Proc Natl Acad Sci USA 2000; 97: 3352-3357.
18 Moser A R, Pitot H C, Dove W F. A dominant mutation that predisposes to multiple intestinal neoplasia in the mouse. Science 1990; 247: 322-324.
19 Washington K, Zemper A E D. Apc-related models of intestinal neoplasia: a brief review for pathologists. Surgical and Experimental Pathology 2019; 2.
20 Moser A R, Luongo C, Gould K A, McNeley M K, Shoemaker A R, Dove W F. ApcMin: a mouse model for intestinal and mammary tumorigenesis. Eur J Cancer 1995; 31A: 1061-1064.
21 Heyer J, Yang K, Lipkin M, Edelmann W, Kucherlapati R. Mouse models for colorectal cancer. Oncogene 1999; 18: 5325-5333.
22 Zhang J, Cao H, Zhang B, Cao H, Xu X, Ruan H et al. Berberine potently attenuates intestinal polyps growth in ApcMin mice and familial adenomatous polyposis patients through inhibition of Wnt signalling. J Cell Mol Med 2013; 17: 1484-1493.
23 Zilberberg A, Lahav L, Rosin-Arbesfeld R. Restoration of APC gene function in colorectal cancer cells by aminoglycoside- and macrolide-induced read-through of premature termination codons. Gut 2010; 59: 496-507.
24 Caspi M, Firsow A, Rajkumar R, Skalka N, Moshkovitz I, Munitz A et al. A flow cytometry-based reporter assay identifies macrolide antibiotics as nonsense mutation read-through agents. J Mol Med (Berl) 2016; 94: 469-482.
Kariv R, Caspi M, Fliss-Isakov N, Shorer Y, Shor Y, Rosner G et al. Resorting the function of the colorectal cancer gatekeeper adenomatous polyposis coli. Int J Cancer 2020; 146: 1064-1074.
26 Keeling K M, Lanier J, Du M, Salas-Marco J, Gao L, Kaenjak-Angeletti A et al. Leaky termination at premature stop codons antagonizes nonsense-mediated mRNA decay in S. cerevisiae. RNA 2004; 10: 691-703.
27 McCaughan K K, Brown C M, Dalphin M E, Berry M J, Tate W P. Translational termination efficiency in mammals is influenced by the base following the stop codon. Proc Natl Acad Sci USA 1995; 92: 5431-5435.
28 Guo D, Cai Y, Chai D, Liang B, Bai N, Wang R. The cardiotoxicity of macrolides: a systematic review. Pharmazie 2010; 65: 631-640.
29 Li X, Wang M, Liu G, Zhou L, Wang Z, Li C. Macrolides use and the risk of sudden cardiac death. Expert Rev Ani Infect Ther 2016; 14: 535-537.
30 Brinker A D, Wassel R T, Lyndly J, Serrano J, Avigan M, Lee W M et al. Telithromycin-associated hepatotoxicity: Clinical spectrum and causality assessment of 42 cases. Hepatology 2009; 49: 250-257.
31 Chlipala E, Bendzinski C M, Chu K, Johnson J I, Brous M, Copeland K et al. Optical density-based image analysis method for the evaluation of hematoxylin and eosin staining precision. J Histotechnol 2020; 43: 29-37.
32 Horai Y, Akatsuka A, Mizukawa M, Nilshina H, Nishikawa S, Ono Y et al. Current status and prospects for quantitative analysis of digital image of pathological specimen using image processing software including artificial intelligence. Translational and Regulatory Sciences 2020; 2: 72-79.
33 Lee H L, Dougherty J P. Pharmaceutical therapies to recode nonsense mutations in inherited diseases. Pharmacol Ther 2012; 136: 227-266.
34 Stamos J L, Weis W I. The beta-catenin destruction complex. Cold Spring Harb Perspect Biol 2013; 5: a007898.
Fredericks E, Dealtry G, Roux S. beta-Catenin Regulation in Sporadic Colorectal Carcinogenesis: Not as Simple as APC. Can J Gastroenterol Hepatol 2018; 2018: 4379673.
36 Davies M L, Roberts G T, Stuart N, Wakeman J A. Analysis of a panel of antibodies to APC reveals consistent activity towards an unidentified protein. Br J Cancer 2007; 97: 384-390.
37 Lecarpentier Y, Schussler O, Hebert J L, Vallee A. Multiple Targets of the Canonical WNT/beta-Catenin Signaling in Cancers. Front Oncol 2019; 9: 1248.
38 Kirst H A, Sides G D. New directions for macrolide antibiotics: pharmacokinetics and clinical efficacy. Antimicrob Agents Chemother 1989; 33: 1419-1422.
39 Kettunen H L, Kettunen A S, Rautonen N E. Intestinal immune responses in wild-type and Apcmin/+ mouse, a model for colon cancer. Cancer Res 2003; 63: 5136-5142.
40 Floquet C, Rousset J P, Bidou L. Readthrough of premature termination codons in the adenomatous polyposis coli gene restores its biological activity in human cancer cells. PLoS One 2011; 6: e24125.
41 Bidou L, Bugaud O, Belakhov V, Baasov T, Namy O. Characterization of new-generation aminoglycoside promoting premature termination codon readthrough in cancer cells. RNA Biol 2017; 14: 378-388.
42 Tian X, Liu Z, Niu B, Zhang J, Tan T K, Lee S R et al. E-cadherin/beta-catenin complex and the epithelial barrier. J Biomed Biotechnol 2011; 2011: 567305.
43 Vazquez-Laslop N, Mankin A S. How Macrolide Antibiotics Work. Trends Biochem Sci 2018; 43: 668-684.
44 Sothiselvam S, Neuner S, Rigger L, Klepacki D, Micura R, Vazquez-Laslop N et al. Binding of Macrolide Antibiotics Leads to Ribosomal Selection against Specific Substrates Based on Their Charge and Size. Cel/Rep 2016; 16: 1789-1799.
45 Hajimirzaei N, Khalili N P, Boroumand B, Safari F, Pourhosseini A, Judi-Chelan R et al. Comparative Study of the Effect of Macrolide Antibiotics Erythromycin, Clarithromycin, and Azithromycin on the ERG1 Gene Expression in H9c2 Cardiomyoblast Cells. Drug Res (Stuttg) 2020; 70: 341-347.
46 Woodhead J L, Yang K, Oldach D, MacLauchlin C, Fernandes P, Watkins P B et al. Analyzing the Mechanisms Behind Macrolide Antibiotic-Induced Liver Injury Using Quantitative Systems Toxicology Modeling. Pharm Res 2019; 36: 48.
47 Morgan R E, Trauner M, van Staden C J, Lee P H, Ramachandran B, Eschenberg M et al. Interference with bile salt export pump function is a susceptibility factor for human liver injury in drug development. Toxicol Sci 2010; 118: 485-500.

### EQUIVALENTS AND SCOPE

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set in verbatim herein herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating familial adenomatous polyposis (FAP) in a patient in need of such treatment, comprising administering to the patient a therapeutically effective amount of a compound of Formula I: 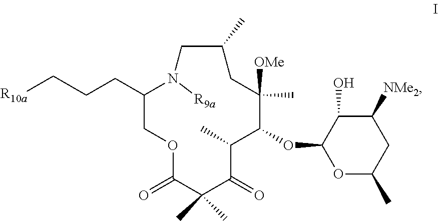
or a pharmaceutically acceptable salt thereof, wherein
R₉ₐ is selected from a group consisting of H and optionally substituted C₁₋₆ alkyl; and
R₁₀ₐ is an optionally substituted heteroaryl.

2. The method according to claim 1, wherein R₉ₐ is methyl, ethyl, propyl, isopropyl, butyl, isobutyl, pentyl, or isopentyl.

3. The method according to claim 1, wherein R₁₀ₐ is:

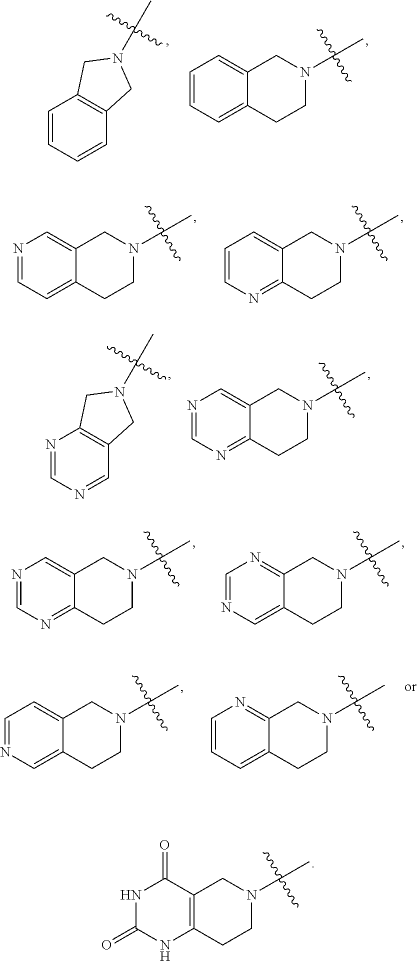

4. The method according to claim 1, wherein the treatment comprises administering to the patient a therapeutically effective amount of a compound of Formula II: 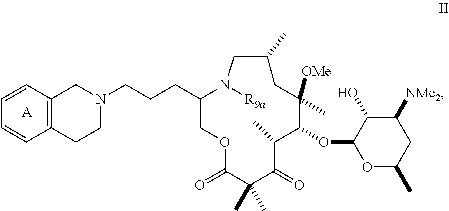
or a pharmaceutically acceptable salt thereof, wherein
R₉ₐ is selected from a group consisting of H or optionally substituted C₁₋₆ alkyl; and 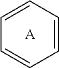
is an optionally substituted 6-membered aromatic or heteroaromatic ring.

5. The method according to claim 4, wherein R₉ₐ is methyl, ethyl, propyl, isopropyl, butyl, isobutyl, pentyl, or isopentyl.

6. The method of claim 4, wherein

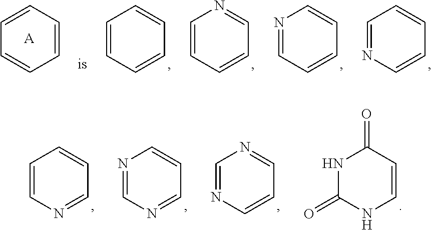

7. The method of claim 1, wherein the compound of Formula I or a pharmaceutically acceptable salt thereof is chosen from a compound depicted below:

| Cmpd # | Structure |
|---|---|
| 1 | 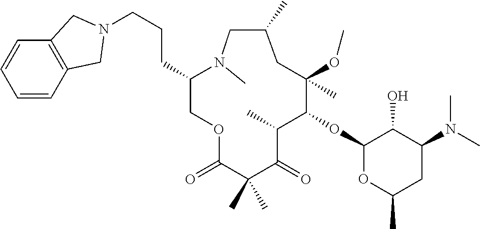 |
| 2 | 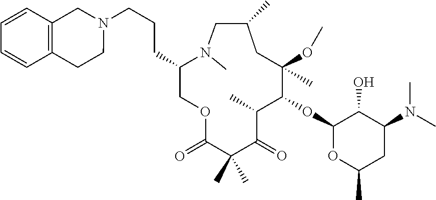 |
| 3 | 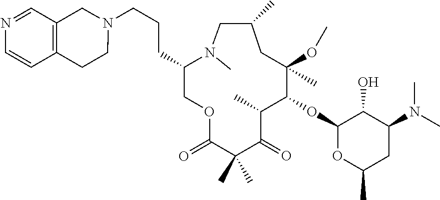 |
| 4 | 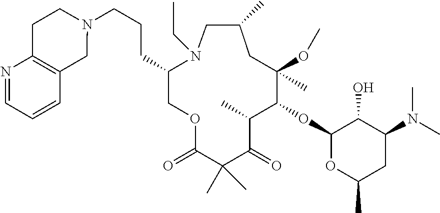 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncccc2C1] |
| 5 | 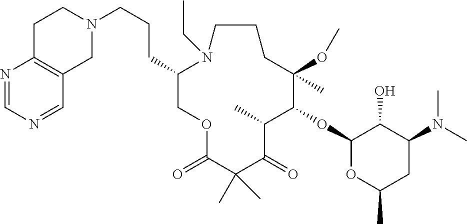 [SMILES: CCN1CCC[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1] |
| 6 | 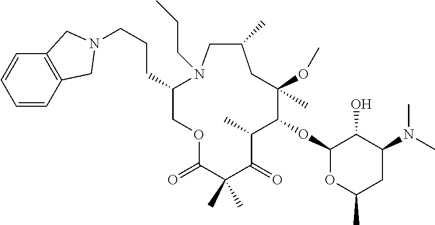 |
| 7 | 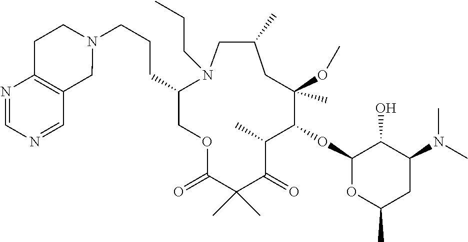 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1] |
| 8 | 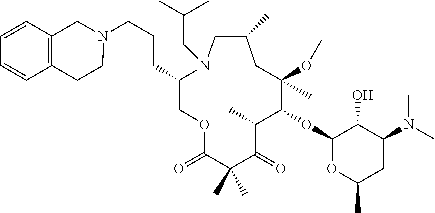 |
| 9 | 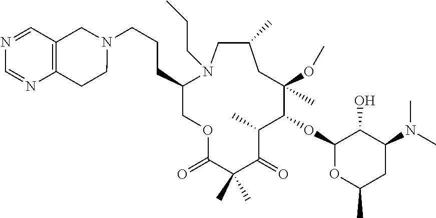 |
| 10 | 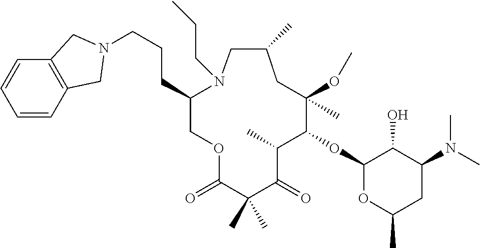 |
| 11 | 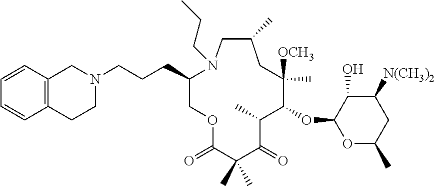 |
| 12 | 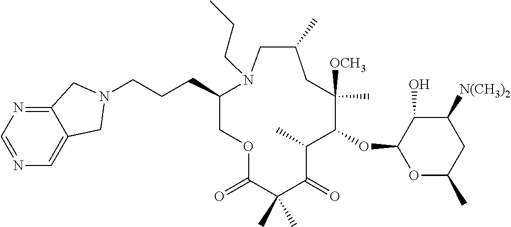 |
| 13 | 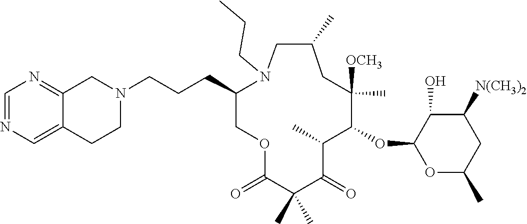 |
| 14 | 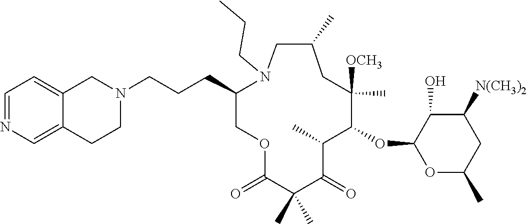 |
| 15 | 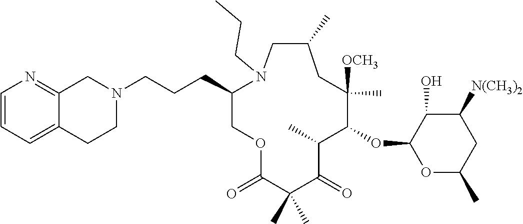 |
| 18 | 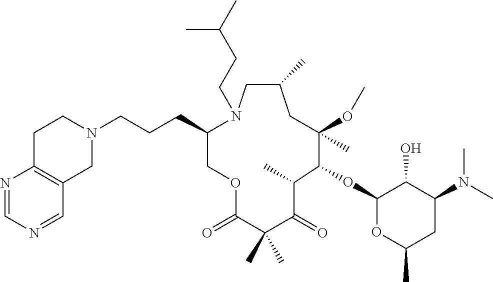 |
| 19 | 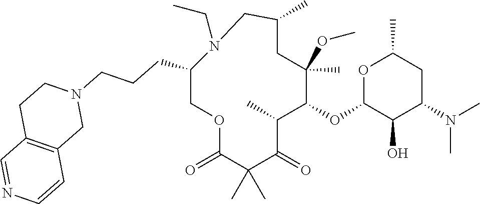 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2cnccc2C1] |
| 20 | 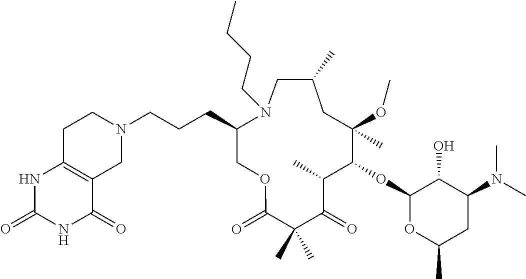 |

8. The method of claim 1, wherein the FAP is FAP, attenuated FAP, or autosomal recessive FAP.

9. The method of claim 8, wherein the FAP is FAP.

10. The method of claim 1, wherein the treatment comprises administering to the patient a pharmaceutical composition comprising a therapeutically effective amount of the compound of Formula I.
